SEQUENCE LISTING (63 sequences)

SEQ ID NO: 1 - protein, 6 aa, Artificial Sequence
  The sequence has been designed and synthesized
MRFGKR

SEQ ID NO: 2 - protein, 31 aa, Artificial Sequence
  The sequence has been designed and synthesized
NSKLHRGGGRSRTSGSTGSMASHARGSPGLQ

SEQ ID NO: 3 - protein, 5 aa, Artificial Sequence
  The sequence has been designed and synthesized
GRELF

SEQ ID NO: 4 - protein, 5 aa, Artificial Sequence
  The sequence has been designed and synthesized
ANELF

SEQ ID NO: 5 - protein, 54 aa, Artificial Sequence
  The sequence has been designed and synthesized
VPAPGETRACGRKLISLVMAVCGDLCNPQEGKDIATECCGNQCSDDYIRSACCP

SEQ ID NO: 6 - protein, 21 aa, Artificial Sequence
  The sequence has been designed and synthesized
SNAELINGLIGMDLGKLSAVG

SEQ ID NO: 7 - protein, 21 aa, Artificial Sequence
  The sequence has been designed and synthesized
SNAELINGLLSMNLNKLSGAG

SEQ ID NO: 8 - protein, 12 aa, Artificial Sequence
  The sequence has been designed and synthesized
EDGNAPFGTMKF

SEQ ID NO: 9 - protein, 13 aa, Artificial Sequence
  The sequence has been designed and synthesized
STVDSSEPVIRDQ

SEQ ID NO: 10 - protein, 8 aa, Artificial Sequence
  The sequence has been designed and synthesized
SPLGTMRF

SEQ ID NO: 11 - protein, 8 aa, Artificial Sequence
  The sequence has been designed and synthesized
NPLGTMRF

SEQ ID NO: 12 - protein, 8 aa, Artificial Sequence
  The sequence has been designed and synthesized
TPLGTMRF

SEQ ID NO: 13 - protein, 13 aa, Artificial Sequence
  The sequence has been designed and synthesized
NPENDTPFGTMRF

SEQ ID NO: 14 - protein, 5 aa, Artificial Sequence
  The sequence has been designed and synthesized
NPEND

SEQ ID NO: 15 - protein, 10 aa, Artificial Sequence
  The sequence has been designed and synthesized
SAEPFGTMRF

SEQ ID NO: 16 - DNA, 184 bp, Artificial Sequence
  The sequence has been designed and synthesized
ggggacaagtttgtacaaaaaagcaggctggatgcgctttggaaaacgtaattcgagctccaccgcggtggcggccgctctagaactagtggatccaccggttccatggctagccacgcgcgtggatcccccgggctgcaggaaacgtaaataacacccagctttcttgtacaaagtggtcccc

SEQ ID NO: 17 - DNA, 184 bp, Artificial Sequence
  The sequence has been designed and synthesized
ggggaccactttgtacaagaaagctgggtgttatttacgtttcctgcagcccgggggatccacgcgcgtggctagccatggaaccggtggatccactagttctagagcggccgccaccgcggtggagctcgaattacgttttccaaagcgcatccagcctgcttttttgtacaaacttgtcccc

SEQ ID NO: 18 - DNA, 112 bp, Artificial Sequence
  The sequence has been designed and synthesized
ggggacaagtttgtacaaaaaagcaggctggatgcgctttggaaaacgtagaggacgagaacttttcggaaaacgtaaataacacccagctttcttgtacaaagtggtcccc

SEQ ID NO: 19 - DNA, 112 bp, Artificial Sequence
  The sequence has been designed and synthesized
ggggaccactttgtacaagaaagctgggtgttatttacgttttccgaaaagttctcgtcctctacgttttccaaagcgcatccagcctgcttttttgtacaaacttgtcccc

SEQ ID NO: 20 - DNA, 112 bp, Artificial Sequence
  The sequence has been designed and synthesized
ggggacaagtttgtacaaaaaagcaggctggatgcgctttggaaaacgtcgtgccaatgaacttttcggtaaacgtaaataacacccagctttcttgtacaaagtggtcccc

SEQ ID NO: 21 - DNA, 112 bp, Artificial Sequence
  The sequence has been designed and synthesized
ggggaccactttgtacaagaaagctgggtgttatttacgtttaccgaaaagttcattggcacgacgttttccaaagcgcatccagcctgcttttttgtacaaacttgtcccc

SEQ ID NO: 22 - DNA, 33 bp, Artificial Sequence
  The sequence has been designed and synthesized
caatgccacgagcaagtagtgttccagcaccag

SEQ ID NO: 23 - DNA, 33 bp, Artificial Sequence
  The sequence has been designed and synthesized
ctggtgctggaacactacttgctcgtggcattg

SEQ ID NO: 24 - DNA, 151 bp, Artificial Sequence
  The sequence has been designed and synthesized
ggggacaagtttgtacaaaaaagcaggctgggttcagttcgtaaaacgcagcaacgccgagcttatcaacggactcatcggaatggatttgggaaaattgtcagctgtcggaaaacgctgacacccagctttcttgtacaaagtggtcccc

SEQ ID NO: 25 - DNA, 151 bp, Artificial Sequence
  The sequence has been designed and synthesized
ggggaccactttgtacaagaaagctgggtgtcagcgttttccgacagctgacaattttcccaaatccattccgatgagtccgttgataagctcggcgttgctgcgttttacgaactgaacccagcctgcttttttgtacaaacttgtcccc

SEQ ID NO: 26 - DNA, 151 bp, Artificial Sequence
  The sequence has been designed and synthesized
ggggacaagtttgtacaaaaaagcaggctgggttcagttcgtaaaacgctcaaacgcggaacttatcaacggtcttctcagcatgaacctcaacaaattgtctggagctggtcgacgatgacacccagctttcttgtacaaagtggtcccc

SEQ ID NO: 27 - DNA, 151 bp, Artificial Sequence
  The sequence has been designed and synthesized
ggggaccactttgtacaagaaagctgggtgtcatcgtcgaccagctccagacaatttgttgaggttcatgctgagaagaccgttgataagttccgcgtttgagcgttttacgaactgaacccagcctgcttttttgtacaaacttgtcccc

SEQ ID NO: 28 - protein, 31 aa, Artificial Sequence; identical to SEQ ID NO: 2
  The sequence has been designed and synthesized

SEQ ID NO: 29 - protein, 5 aa, Artificial Sequence
  The sequence has been designed and synthesized
GRELF

SEQ ID NO: 30 - protein, 5 aa, Artificial Sequence
  The sequence has been designed and synthesized
ANELF

SEQ ID NO: 31 - protein, 54 aa, Artificial Sequence; identical to SEQ ID NO: 5
  The sequence has been designed and synthesized

SEQ ID NO: 32 - protein, 29 aa, Artificial Sequence
  The sequence has been designed and synthesized
VQFVKRSNAELINGLIGMDLGKLSAVGKR

SEQ ID NO: 33 - protein, 29 aa, Artificial Sequence
  The sequence has been designed and synthesized
VQFVKRSNAELINGLLSMNLNKLSGAGRR

SEQ ID NO: 34 - DNA, 32 bp, Artificial Sequence
  The sequence has been designed and synthesized
gactctgcagcatttccaagacacatttgacg

SEQ ID NO: 35 - DNA, 29 bp, Artificial Sequence
  The sequence has been designed and synthesized
gactggatccttttccaaagcgcatggtt

SEQ ID NO: 36 - DNA, 20 bp, Artificial Sequence
  The sequence has been designed and synthesized
gtttgttttccggcactttc

SEQ ID NO: 37 - DNA, 48 bp, Artificial Sequence
  The sequence has been designed and synthesized
agtcgacctgcaggcatgcaagctaattgaacttggaatcgtggtagt

SEQ ID NO: 38 - DNA, 20 bp, Artificial Sequence
  The sequence has been designed and synthesized
cggcactttcctcatttttc

SEQ ID NO: 39 - DNA, 20 bp, Artificial Sequence
  The sequence has been designed and synthesized
accgatttctgatcgacgtg

SEQ ID NO: 40 - DNA, 45 bp, Artificial Sequence
  The sequence has been designed and synthesized
cagcagtttccctgaattaaaattaaacaattgccggagcttttc

SEQ ID NO: 41 - DNA, 20 bp, Artificial Sequence
  The sequence has been designed and synthesized
ttctgatcgacgtgttggtt

SEQ ID NO: 42 - DNA, 24 bp, Artificial Sequence
  The sequence has been designed and synthesized
agcttgcatgcctgcaggtcgact

SEQ ID NO: 43 - DNA, 25 bp, Artificial Sequence
  The sequence has been designed and synthesized
aagggcccgtacggccgactagtag

SEQ ID NO: 44 - DNA, 27 bp, Artificial Sequence
  The sequence has been designed and synthesized
ggaaacagttatgtttggtatattggg

SEQ ID NO: 45 - DNA, 25 bp, Artificial Sequence
  The sequence has been designed and synthesized
taattttaattcagggaaactgctg

SEQ ID NO: 46 - DNA, 24 bp, Artificial Sequence
  The sequence has been designed and synthesized
aaagttggaaacagttatgtttgg

SEQ ID NO: 47 - DNA, 185 bp, Artificial Sequence
  The sequence has been designed and synthesized
ggggacaagtttgtacaaaaaagcaggctggatgcgctttggaaaacgtaattcgaagctccaccgcggtggcggccgctctagaactagtggatccaccggttccatggctagccacgcgcgtggatcccccgggctgcaggaaacgtaaataacacccagctttcttgtacaaagtggtcccc

SEQ ID NO: 48 - DNA, 118 bp, Artificial Sequence
  The sequence has been designed and synthesized
ggggacaagtttgtacaaaaaagcaggctggatgcgctttggaaaacgttctccactgggaacaatgcgctttggcaaacgtaaataacacccagctttcttgtacaaagtggtcccc

SEQ ID NO: 49 - DNA, 118 bp, Artificial Sequence
  The sequence has been designed and synthesized
ggggacaagtttgtacaaaaaagcaggctggatgcgctttggaaaacgtactccattgggaactatgcgttttggaaaacgtaaataacacccagctttcttgtacaaagtggtcccc

SEQ ID NO: 50 - DNA, 118 bp, Artificial Sequence
  The sequence has been designed and synthesized
ggggacaagtttgtacaaaaaagcaggctggatgcgctttggaaaacgtaaccctcttggaaccatgcgctttggaaaacgtaaataacacccagctttcttgtacaaagtggtcccc

SEQ ID NO: 51 - DNA, 133 bp, Artificial Sequence
  The sequence has been designed and synthesized
ggggacaagtttgtacaaaaaagcaggctggatgcgctttggaaaacgtaatcctgagaacgacacaccattcggaacaatgagatttggaaaacgtaaataacacccagctttcttgtacaaagtggtcccc

SEQ ID NO: 52 - DNA, 130 bp, Artificial Sequence
  The sequence has been designed and synthesized
ggggacaagtttgtacaaaaaagcaggctggatgcgctttggaaaacgttctactgttgattcttcggagcccgtcattcgtgatcagaaacgtaaataacacccagctttcttgtacaaagtggtcccc

SEQ ID NO: 53 - DNA, 124 bp, Artificial Sequence
  The sequence has been designed and synthesized
ggggacaagtttgtacaaaaaagcaggctggatgcgctttggaaaacgtagtgcagagccattcggtactatgcgttttggaaaacgtaaataacacccagctttcttgtacaaagtggtcccc

SEQ ID NO: 54 - DNA, 102 bp, Artificial Sequence
  The sequence has been designed and synthesized
gcaggctggatgcgctttggaaaacgtagtgcaactccattcggtactatgcgttttggaaaacgtaaataacacccagctttcttgtacaaagtggtcccc

SEQ ID NO: 55 - DNA, 25 bp, Artificial Sequence
  The sequence has been designed and synthesized
ggtatctgtggtttatgtcggatag

SEQ ID NO: 56 - DNA, 21 bp, Artificial Sequence
  The sequence has been designed and synthesized
gaacggcatacgttcaggata

SEQ ID NO: 57 - DNA, 21 bp, Artificial Sequence
  The sequence has been designed and synthesized
aaactaccctgtgcgattagg

SEQ ID NO: 58 - DNA, 22 bp, Artificial Sequence
  The sequence has been designed and synthesized
cttccatacgtcaagcactttc

SEQ ID NO: 59 - DNA, 20 bp, Artificial Sequence
  The sequence has been designed and synthesized
ccctcaatgtctctacttgt

SEQ ID NO: 60 - DNA, 22 bp, Artificial Sequence
  The sequence has been designed and synthesized
ttcctcgtgctttacggtatcg

SEQ ID NO: 61 - DNA, 21 bp, Artificial Sequence
  The sequence has been designed and synthesized
actttcaggcgaacacatact

SEQ ID NO: 62 - DNA, 30 bp, Artificial Sequence
  The sequence has been designed and synthesized
tcataattgtttgtttgacaaaaaccggga

SEQ ID NO: 63 - DNA, 30 bp, Artificial Sequence
  The sequence has been designed and synthesized
ggtggaaacggaaatgaaagaaaaaaccga

(19) United States
(12) Patent Application Publication
(10) Pub. No.: US 2022/0380430 A1
(43) Pub. Date: Dec. 1, 2022

(54) Non-Transgenic Functional Rescue of Neuropeptides

(71) Applicant: Worcester Polytechnic Institute, Worcester, MA (US)

(72) Inventors: Jagan Srinivasan, Shrewsbury, MA (US); Douglas Reilly, Marlborough, MA (US); Elizabeth DiLoreto, Jefferson, MA (US)

(21) Appl. No.: 17/749,555

(22) Filed: May 20, 2022

Related U.S. Application Data
(60) Provisional application No. 63/191,046, filed on May 20, 2021.

(51) Int. Cl. C07K 14/575; A61K 31/7105; C12N 15/70; G01N 33/68; A61K 35/74

(52) U.S. Cl. CPC C07K 14/57545; A61K 31/7105; C12N 15/70; G01N 33/68; A61K 35/74; A61K 38/00

(57) ABSTRACT

Kits and methods for rescuing at least one neuropeptide in a subject are described herein, including identifying at least one neuropeptide and recombining a nucleic acid sequence of the neuropeptide to obtain a recombinant nucleic acid neuropeptide; cloning the recombinant nucleic acid neuropeptide into a plasmid to obtain a recombinant neuropeptide plasmid and transforming the recombinant neuropeptide plasmid into a bacterial cell to obtain a transformed neuropeptide bacterial feed; and feeding the bacterial feed to the subject thereby rescuing the neuropeptide in the subject.

### RELATED APPLICATION

This application claims the benefit of priority to U.S. provisional application No. 63/191,046 filed May 20, 2021, having the title, “Non-Transgenic Functional Rescue of Neuropeptides” by inventors, Jagan Srinivasan, Douglas Reilly, and Elizabeth DiLoreto, which is hereby incorporated by reference herein in its entirety.

### GOVERNMENT SUPPORT

This invention was made with government support under grant numbers 1R01DC016058 awarded by the National Institutes of Health. The government has certain rights in the invention.

### BACKGROUND

Organisms upon presentation with a host of environmental cues sense, interpret, and enact appropriate responses to stimuli. The interpretation and integration of stimuli is a dynamic process that requires neural circuits to be flexible to elicit the proper behavior. For example, a single stimulus can drive multiple reactions depending on internal or environmental states of the organism. In the roundworm Caenorhabditis elegans, the same small molecular cue (ascr #3) upon presentation elicits a different reaction from the male and the hermaphrodite roundworm. The male roundworms are attracted to the cue and the hermaphrodite roundworms avoid the cue. Indole elicits different reactions depending on the amount present in the environment. At low concentrations, indole is attractive with a pleasant, floral aroma, though at high concentrations, it is repulsive, smelling pungent, reminiscent of feces or rot. To modulate competing behaviors in response to the same cue, for example, different neurons can be involved at the circuit level or changes can occur at the synaptic level. A more dynamic approach for modulating neural responses is peptidergic signaling.

Neuropeptides are short amino acid chains that serve to regulate neural circuits, and function as neurotransmitters and neurohormones. Neuropeptides signal on longer time scales than, though often in concert with neurotransmitters to regulate synaptic activity, typically through G-protein coupled receptor signaling cascades. Multiple modulators allow neurons to serve unique functions within discrete neural circuits. Neuropeptides serve a broad array of functions across the animal kingdom. Oxytocin-like and vasopressin-like peptides are neurohormones that regulate social attachment, lactation, and blood pressure by contracting muscles in mammals, dating back 600-700 million years. However, in the Echinoderm Sea star, Asterias rubens, vasopressin-like and oxytocin-like neuropeptide 4 ortholog asterotocin serves instead to relax muscles in the cardiac stomach during fictive feeding. The C. elegans vasopressin ortholog, nematocin, interacts with serotonin and dopamine signaling to modulate gustatory associative memory and male mating behaviors.

C. elegans are a microscopic nematode that displays robust behaviors driven by just over 300 neurons. The C. elegans genome encodes three classes of neuropeptides: FMRFamide-like peptides (FLP), insulin-like peptides (INS), and non-FLP/insulin neuropeptide-like peptides (NLP), encoding over 300 individual neuropeptides through 131 genes that modulate the functional connectome. The complexity of the neuropeptide genome, combined with extra-synaptic neuropeptides signaling makes elucidating the role of individual peptides difficult, as canonical studies often rely on null mutations and transgenic rescues. As such, these studies are often incomplete, as full gene rescue restores complete preproproteins and makes discrimination of discrete peptide function impossible.

Therefore, there is a need for an assay that rescues neuropeptides synthesized endogenously within the subject genome.

### SUMMARY

Current methods of genetic rescue in Caenorhabditis elegans are not ideal for understanding the function of individual peptides; whether by cost inhibition or equipment limitations or due to whole gene rescue not being ideal for studying discrete peptides. Feeding of peptides via E. coli has proven successful in manipulating C. elegans biological function.

The use of Gateway Cloning to develop expression vectors allows for the development of high-throughput experiments targeting individual peptides, or combinations thereof. Additionally, this technique is readily accessible compared to traditional transgenic rescue approaches. The paradigm rescues individual, processed neuropeptides, leveraging the genetic amenability of the C. elegans food source, Escherichia coli, to circumvent the need for transgenic development and enable high-throughput rescue and elucidation of individual neuropeptide function.

An aspect of the invention described herein provides a method for high throughput screening for elucidating function of at least one neuropeptide, the method including: identifying the at least one neuropeptide and recombining a nucleic acid sequence of the neuropeptide to obtain a recombinant nucleic acid neuropeptide; cloning the recombinant nucleic acid neuropeptide into a plasmid to obtain a recombinant neuropeptide plasmid and transforming the recombinant neuropeptide plasmid into a bacterial cell to obtain a transformed neuropeptide bacterial feed; and feeding the bacterial feed to a subject and observing response of the subject to at least one stimulus thereby elucidating function of the neuropeptide.

In an embodiment of the method, recombining further includes adding cleavage sites at 5′ end and 3′ end of the nucleic acid sequence of the neuropeptide. In an embodiment of the method, the plasmid further includes a promoter sequence before the nucleic acid sequence of the neuropeptide. An embodiment of the method further includes rescuing the subject from loss of function by feeding the neuropeptide bacterial feed.

In an embodiment of the method, feeding further includes delivering mRNA to the subject. An embodiment of the method further includes after feeding, translating the mRNA to neuropeptide in the subject. In an embodiment of the method, the bacterial cell is at least one Escherichia coli strain selected from: DH5α, and OP50. In an embodiment of the method, the plasmid is at least one selected from pDEST-527, and pL4440.

An embodiment of the method further includes recombining a control sequence, cloning the control sequence in another plasmid, transforming the plasmid into bacterial cell, and feeding the bacterial cell to the subject as a negative control. For example the control sequence is SCRAMBLE sequence.

In an embodiment of the method, the neuropeptide is at least one selected from: TRH-1A, TRH-1B, INS-6, PDF-1A, PDF-1B, flp-3, npr-10, frpr-16, GFP, and FLP.

An aspect of the invention described herein provides a method for rescuing subject by loss of function of at least one neuropeptide, the method including: identifying the at least one neuropeptide and recombining a nucleic acid sequence of the neuropeptide to obtain a recombinant nucleic acid neuropeptide; cloning the recombinant nucleic acid neuropeptide into a plasmid to obtain a recombinant neuropeptide plasmid and transforming the recombinant neuropeptide plasmid into a bacterial cell to obtain a transformed neuropeptide bacterial feed; and feeding the bacterial feed to the subject thereby rescuing the subject from loss of function of the neuropeptide.

An aspect of the invention described herein provides a kit to validate a neuropeptide function using a rescue by feeding assay, the kit including: a bacterial feed comprising bacterial cells transformed with a recombinant nucleic acid sequence of the neuropeptide cloned in a plasmid; instructions for use; and a container.

### BRIEF DESCRIPTION OF DRAWINGS

FIG. 1A-FIG. 1B is a set of schematic drawings of an overview of the Rescue-By-Feeding Technique to Rescue Neuropeptide Function. FIG. 1A illustrates design of an expression vector containing the peptide sequence of interest. The vector sequence includes Gateway Cloning sites attR1 and attR2, a 6×-histidine tag (6×-His), and MRFGKR (SEQ ID NO: 1) and KRK-STOP sequences flanking the peptide sequence of interest. The vector is transformed into E. coli DH5a cells allowing for the expression of peptides. The bacteria expressing the neuropeptides is then fed to neuropeptide loss-of-function worms and assayed for rescue of behavior. FIG. 1B illustrates different assays used to quantify behavioral activity. Three different assays were utilized herein for the quantification of rescue; (1) Size Comparison, (2) Chemotaxis and (3) Excursion Assay. For Size Comparison, mutant worms are fed the peptide for 48 hours before assaying for body morphology on an automated worm tracker. In the second assay, Chemotaxis, an NaCl gradient chemotaxis assay is used to observe salt attraction of worms of rescued neuropeptides. The Excursion Assay quantified the ability for males to leave a food source in search of mates. Minor Excursion denotes worms that did not leave the food source. Major Excursion denotes worms that left the food source in search of mates, indicating rescue. The sequence listing material in computer readable form ASCII text file (18 kilobytes) created 7/22/2022 entitled “WPI21-11_Sequence_Listing_05202022”, containing sequence listing numbers 1-63, has been electronically filed herewith and is incorporated by reference herein in its entirety.

FIG. 2A-FIG. 2B are a set of graphs depicting that feeding of TRH-1 Peptides rescues the body volume defects in trh-1 Mutants. FIG. 2A shows that trh-1 mutant worms exhibit body volume defects. The trh-1 mutants show a reduced body volume compared to wild-type worms upon feeding E. coli OP50 (Mann-Whitney test, ** p=0.0012). FIG. 2B shows that feeding of TRH-1 peptides results in restoration of body volume in trh-1 lof worms. The trh-1 mutant worms fed with scramble peptide, display significant increase in body volume feeding compared to wild-type animals. The trh-1 mutants fed either TRH-1A or TRH-1B or even a combination of both exhibit a restoration of wild-type body volume. Error bars denote SEM. n values denoted in graph. Kruskal-Wallis followed by Dunn's multiple comparisons, **/##p<0.01, ####p<0.0001, 26 *denote mutant worms compared to wild-type control, #denote mutant worm condition compared to mutant worms fed scramble peptide.

FIG. 3A-FIG. 3B is a set of bar graphs showing that chemotaxis defects of ins-6 neuropeptide mutants are rescued by feeding the INS-6 Peptide. FIG. 3A shows chemotaxis index for 750 mM NaCl of wild-type (WT), ins-6 loss of function, and genetic ins-6 rescue (ins-6;Pins-6;ins-6), animals fed E. coli OP50. Animals with loss-of function ins-6 gene exhibit significant decreased attraction and genetic rescue of ins-6 results in a partial rescue of chemotaxis. FIG. 3B shows chemotaxis index of peptide fed worms for 750 mM NaCl. The ins-6 lof worms fed scramble display significantly lower chemotaxis compared to scramble-fed wild type animals. Feeding of INS-6 peptide to ins-6 lof worms results in complete rescue of NaCl chemotaxis. Overexpression of INS-6 does not result in changes in attraction towards 750 mM NaCl. n values are denoted in graphs. Error bars denote SEM. One-Way ANOVA, followed by Bonferroni's Correction * p<0.05, ** p<0.01, *** p<0.001, **** p<0.0001, comparisons noted with line over bars.

FIG. 4A-FIG. 4B is a set of graphs illustrating that pdf-1 lof males fed discrete PDF-1 peptides differentially rescue food-leaving behavior. FIG. 4A shows percent of male worms leaving food across time which is used to visualize the location of the males at different time points. The him-5 males serve as the wild-type worms in this assay compared to males deficient in pdf-1 fed PDF-1 peptide to rescue behavior. At each time point the percent of worm within each boundary is noted (Major Excursion is beyond 10 mm radius of the food (purple), Minor Excursion is worms who left food but stayed within 10 mm (pink), and those remaining on food were classified as Never Left Food (yellow)). FIG. 4B shows the probability of leaving. Comparison of the leaving behavior of the worms is shown as a likelihood of the worms seeking to travel beyond 35 mm from the food. n; him-5 males fed scramble=55, pdf-1 males fed scramble=53, pdf-1 males fed PDF-1A=58, pdf-1 males fed PDF-1B=51, pdf-1 males fed PDF-1A and PDF-1B=27 51). Error bars denote SEM. ANOVA, followed by Bonferroni's Correction, ***/###p<0.001, ####p<0.0001, *denote mutant worms compared to wild-type control, #denote mutant worm condition compared to mutant worms fed scramble peptide.

FIG. 5A-FIG. 5H is a set of graphs showing that TRH-1 peptide feeding affects body length, width, and area of trh-1 mutants. FIG. 5A shows relative body volume of E. coli OP50 fed worms, displaying data points (Mann-Whitney test, ** p=0.0012). FIG. 5B shows relative body volume of peptide worms, all data points wild-type fed scramble, trh-1 fed scramble, TRH-1A, TRH-1B, or both TRH-1A+B. FIG. 5C shows relative body length of peptide fed worms. FIG. 5D shows relative body length of peptide fed worms, all data points. FIG. 5E shows relative body width of worms fed peptide. FIG. 5F shows relative body width of peptide fed worms, all data points. FIG. 5G shows relative body area of peptide fed worms. FIG. 5G shows relative body area of peptide fed worms displaying all data points. n for all panels wild-type=454, trh-1 fed scramble=559, fed TRH-1A=483, fed TRH-1B=441, fed TRH-1A+B=221. Error bars denote SEM. (b-g) Kruskal-Wallis followed by Dunn's multiple comparison tests, */#p<0.05, **/##, p<0.01, ***/###p<0.001, ****/####p<0.0001, *denote mutant worms compared to wild-type control, #denote mutant worm condition compared to mutant worms fed scramble peptide.

FIG. 6A-FIG. 6B is a set of graphs showing chemotaxis index of partially rescued ins-6 lof worms to 750 mM NaCl without and with peptide feeding. FIG. 6A shows Chemotaxis Index to 750 mM NaCl of wild-type, ins-6 rescued specifically in ASI neurons and a complete genetic rescue of ins-6 animals. Rescue of ins-6 in ASI neurons does not result in NaCl chemotaxis, suggesting that this neuron does not play a role in this behavior. FIG. 6B shows Chemotaxis Index of INS-6 peptide-fed wild-type and genetically rescued ins-6 worms. Overexpression of INS-6 by feeding does not affect chemotaxis in wild type animals and the different rescue lines of ins-6. n values denoted in graphs. Error bars denote SEM. One-Way ANOVA, followed by Bonferroni's Correction. * p<0.05, ** p<0.01.

FIG. 7A-FIG. 7B is a set of graphs leaving behavior of him-5 and pdf-1 males fed with PDF-1 peptides. FIG. 7A shows percentage of him-5 males leaving food after being fed PDF-1 peptides on E. coli OP50. PDF-1B feeding results in complete rescue of food-leaving behavior of pdf-1 lof males at all three timepoints. FIG. 7B shows a computation of Probability of leaving for him-5 and pdf-1 males fed E. coli OP50. ANOVA, followed by Bonferroni's Correction, Two-tailed t-test of samples of equal variance. *** p<0.001.

FIG. 8 is a schematic drawing of the final expression vector map. The T7-Promoter sequence (driven by IPTG induction) is included as part of the pDEST-527 backbone. The two peptide processing cleavage sites (MRFGKR (SEQ ID NO: 1) and KR-STOP) are added flanking the peptide sequence at the stage of DNA Oligo synthesis. “Peptide” denotes the coding sequence for the peptide of interest, and is the unique portion of the vector for each rescue.

FIG. 9A-FIG. 9B is a set of graphs illustrating that rescue-by-feeding of FLP-3 restores male attraction. FIG. 9A shows that the him-8 males (wild-type males) do not avoid the cue of interest (shown through a negative Avoidance Index value), while flp-3 mutants do avoid (positive Avoidance Index). Feeding of SCRAMBLE does not affect behavior, while feeding of FLP-3-2 and FLP-3-9 undo the Avoidance phenotype associated with the flp-3 mutation. FIG. 9B shows that the him-8 males are attracted to the cue of interest (shown through a positive “Log(fold-change) A/V” value), while flp-3 males are not attracted (shown through a value that is not significantly different than “0.0”). Feeding of SCRAMBLE does not affect behavior, while feeding of FLP-3-2 seems to make the animals less attracted to the cue (inferred from a more negative value), and feeding by FLP-3-9 restores the attraction to wild-type, or him-8, value.

FIG. 10A-FIG. 10B is a set of graphs illustrating that the rescue-by-feeding of TRH-1 and INS-6 restores wild-type growth and behavior. FIG. 10A shows that N2 (another wild-type strain) animals are set as the 100% relative body volume. The trh-1 mutant animals fed SCRAMBLE peptide are defective in their growth, being almost 120% the N2 body size. Feeding of either TRH-1A, TRH-1B, or a combination of both restores this growth defect back to ˜100% Relative Body Volume. FIG. 10B shows that WT (wild-type) animals exhibit a Chemotaxis Index of approximately 0.7, which drops to less than 0.4 in ins-6 mutants (fed SCRAMBLE peptide). Feeding of INS-6 Peptide restores the Chemotaxis Index at a level similar to the canonical transgenic rescue.

FIG. 11A-FIG. 11E is a schematic drawing and a set of graphs. FIG. 11A shows the design of the Single Worm Attraction Assay (SWAA). The outer 40 wells of a 48-well suspension cell culture plate are seeded with NGM agar and a thin lawn of OP50 E. coli. A random block design results in spatial control (light gray), vehicle control (dark gray), and ascaroside (purple) containing wells. Quadrants are recorded for 15 m. (Inset) The structure of ascr #8. FIG. 11B shows the raw dwell times of males of N2 (blue), him-5 (red), him-8 (purple), osm-3;him-5 (orange) in SWAA. FIG. 11C shows transformed log(fold-change) of male dwell time data. FIG. 11D shows raw dwell time of hermaphrodites across strains and FIG. 11E shows log(fold-change) of SWAA data of hermaphrodites. Light gray denotes spatial controls (“S”) (when applicable), dark grey denotes vehicle controls (“V”), colors denote ascr #8 values (“A”). ♂ denotes male data, [symbol] denotes hermaphrodite data. For all figures: Error bars denote SEM. n≥5. *p<0.05, **p<0.01, ***p<0.001, ****p<0.0001, unless denoted otherwise. For 1b: ++++p<0.0001, vehicle vs. spatial control.

FIG. 12A-FIG. 12L is a set of graphs and a set of microphotographs showing screen of FMRFamide-like peptide (flp) defective mutants in response to ascr #8 in SWAA. FIG. 12A shows male raw dwell time and FIG. 12B shows log(fold-change) values revealed specificity of flp-3 in the ascr #8 behavioral response. FIG. 12C shows that a drop avoidance assay was employed to reveal a change in behavioral valence, with flp-3 males avoiding ascr #8. FIG. 12D shows that hermaphrodite raw dwell times and FIG. 12E shows that log(fold-change) values in response to ascr #8 are consistent across flp mutants. FIG. 12F shows that ascr #8 avoidance in hermaphrodites is unaffected by the loss of flp-3. FIG. 12G and FIG. 12I shows that attractive behavior in response to ascr #8 is restored in flp-3 mutant males by expressing flp-3 under its endogenous promoter. FIG. 12G shows raw dwell times in males and FIG. 12H shows log(fold-change) values), as well as FIG. 12I shows rescue of flp-3 restores avoidance to ascr #8. FIG. 12J-FIG. 12L shows expression pattern of pflp-3::flp-3::mCherry in the male tail. Colocalization with gpa-1::GFP in the SPD spicule neurons suggests that flp-3 is expressed in male-specific neurons in the tail (arrows). Asterisk denotes coelomocyte accumulation of GFP. FIG. 12J shows GFP, FIG. 12K shows mCherry, FIG. 12L shows merged image at ˜×90 magnification. In FIG. 12H, ⋄⋄p<0.01 for flp-3 mutant versus transgenic rescue. Light gray denotes spatial controls (“S”) (when applicable), dark gray denotes vehicle controls (“V”), colors denote ascr #8 values (“A”) (him-8, purple; flp-3, teal; flp mutants, blue; transgenic rescues, light blue). ♂ denotes male data, [symbol] denotes hermaphrodite data.

FIG. 13A-FIG. 13E is a set of graphs and microphotographs illustrating that the G protein-coupled receptor, NPR-10, is required for the male behavioral response to ascr #8. FIG. 13A and FIG. 13B show that male raw dwell time and log(fold-change) values for npr receptor mutants and npr-10 rescue in the SWAA, respectively. FIG. 13C shows that npr-10 males exhibit increased avoidance to ascr #8 compared to the other receptors. Expression of npr-10 under its endogenous promoter rescues the avoidance phenotype. FIG. 13D shows the localization of pnpr-10::npr-10::GFP in the amphid region of the male head. Neurons expressing npr-10 include the inner labial neurons, IL2, and their respective socket cells (ILso), the interneurons RMEV and RMEL, the chemosensory neurons ADL and ASG, as well as the interneurons AVF and AVK. FIG. 13E shows expression of pnpr-10::npr-10::GFP in the mail tail. Localization is observed in the B-class Ray neurons. ♂ denotes male data.

FIG. 14A-FIG. 14h is a set of graphs showing dose response curves. FIG. 14A shows the dose response curve for FLP-3-1. FIG. 14B shows the dose response curve for FLP-3-2. FIG. 14C shows the dose response curve for FLP-3-3. FIG. 14D shows dose response curve for FLP-3-4. FIG. 14E shows dose response curve for FLP-3-5. FIG. 14F shows dose response curve for FLP-3-7. FIG. 14G shows dose response curve for FLP-3-8. FIG. 14H shows dose response curve for FLP-3-9 for activation of NPR-10B (blue circles) and FRPR-16 (red triangles). Peptides FLP-3-6 and FLP-3-10 did not activate either receptor. FIG. 14I shows EC₅₀ values and 95% Confidence Intervals for FLP-3 peptide activating NPR-10B and FRPR-16. Error bars denote SEM. n≥6.

FIG. 15A-FIG. 15F is a set of schematic drawings, graphs and microphotographs showing that FRPR-16 is required for the male behavioral response to ascr #8 FIG. 15A and FIG. 15B show the design of CRISPR/Cas9-mediated frpr-16 null mutation construct. FIG. 15A shows the wild-type gene, with CRISPR cut sites marked, along with 450 bp homology arm regions. FIG. 15B shows the mutant gene sequence, consisting of an inverted cassette driving loxP flanked pmyo-2::GFP and prps-27::neoR expression. FIG. 15C and FIG. 15D shows that frpr-16 lof male animals display loss of attraction to ascr #8 and rescue of frpr-16 under its endogenous promoter rescues the attraction phenotype. frpr-16;npr-10 double mutant animals do not display an additive phenotype in terms of attraction as shown in FIG. 15C Raw dwell time and FIG. 15D log(fold-change) values. FIG. 15E shows that frpr-16 lof male animals display increased avoidance to ascr #8 and rescue of frpr-16 restores avoidance to wild-type male animals. The frpr-16;npr-10 double mutant animals do not display avoidance to ascr #8. FIG. 15F shows the expression pattern of pfrpr-16::frpr-16::SL2::mCherry in male C. elegans at ×20 magnification. Localization within the ventral cord denoted. (inset), Amphid localization of pfrpr-16::frpr-16::SL2::mCherry at ˜×120 within the reverse locomotion command interneurons, AVA, AVE, and AVD, as well as the BAG neuron (anterior to the nerve ring). In FIG. 15D ⋄⋄p<0.01 for frpr-16 lof mutant versus transgenic rescue. ♂ denotes male data.

FIG. 16A-FIG. 16h is a set of schematic drawings and graphs showing that peptide feeding rescues wild-type behavior and reveals two active peptides within the FLP-3 precursor. FIG. 16A is an overview of rescue-by-feeding paradigm: in which the top plasmid is generated which encodes the peptide of interest flanked by EGL-3 cleavage sites, with a 6×-His tag upstream. The flp-3 lof animals are raised on bacteria expressing the FLP-3 peptide of interest for 72-96 h and are assayed as young adults. FIG. 16B shows avoidance behavior of him-8 and flp-3 animals raised on scramble peptide or specified FLP-3 peptides. The him-8 and flp-3 males fed SCRAMBLE display their characteristic avoidance to ascr #8. The flp-3 males fed peptides FLP-3-2 or FLP-3-9 restore avoidance to wild-type levels. FIG. 16C and FIG. 16D shows flp-3 male animals fed FLP-3-9 peptides rescues attraction behavior to ascr #8. FIG. 16C shows raw dwell time and FIG. 16D shows log(fold-change) values for him-8 and flp-3 males fed peptides FLP-3-2 or FLP-3-9. FIG. 16E shows mutational schematic of FLP-3-7 to generate FLP-3-7T: the glutamate (“E”) in position 9 was mutated to a threonine (“T”). FIG. 16F shows that flp-3 lof male animals raised on FLP-3-7T suppress avoidance to ascr #8 suggesting the importance of amino acid threonine in mediating avoidance. FIG. 16G and FIG. 16H show that flp-3 animals raised on FLP-3-7 and FLP-3-7T display no rescue of attractive behavior to ascr #8. FIG. 16G shows raw dwell time and FIG. 16H shows log(fold-change) of flp-3 animals raised on FLP-3-7 and FLP-3-7T. ♂ denotes male data.

FIG. 17A-FIG. 17B are a set of schematic drawings showing that two FLP-3 NP/NPR modules mediate the male behavioral response to ascr #8. FIG. 17A shows sequence specificity of FLP-3 peptide function. FLP-3-1, FLP-3-4, and FLP-3-7 exhibit no sex-specific effects on ascr #8 behavioral response. The threonine in the 9th position of FLP-3-2 and FLP-3-9, and in the mutated FLP-3-7T (red) is required for suppressing the sex-specific avoidance of ascr #8. The NPEND sequence of FLP-3-9 drives male attraction to ascr #8. FIG. 17B shows a schematic of the neural circuit regulating male responses to ascr #8 pheromone. ascr #8 is sensed by the male-specific CEM neurons. The neuropeptide gene flp-3 is expressed in the IL1 neurons in the head and the SPD spicule neurons in the tail (teal). Following release of processed FLP-3 peptides, FLP-3-2 and FLP-3-9 are sensed by NPR-10 and FRPR-16 expressing neurons (blue and red, respectively) to mediate dwelling in the mating pheromone by modulating forward and reverse locomotion. Green neurons are connections inferred from the male synaptic connectome. Gold denotes the command interneuron AVB and the forward locomotory circuitry.

### DETAILED DESCRIPTION

Neuropeptides exert essential functions in animal physiology e.g., reproduction, development, growth, energy homeostasis, cardiovascular activity and stress response. Comparison of cDNA or genomic sequences between distant species has allowed the discovery, in mammals, of several biologically active peptides previously identified in non-mammalian species. However, elucidation of function is not possible at a large-scale for the various neuropeptides identified by genomic approaches. The methods described herein provide a high-throughput screening of function of these peptides.

Animals constantly respond to changes in their environment and internal states via neuromodulation. Neuropeptide genes modulate neural circuits by encoding either multiple copies of the same neuropeptide or different neuropeptides. This architectural complexity makes it difficult to determine the function of discrete and active neuropeptides. The methods described herein provide a novel genetic tool that facilitates functional analysis of individual peptides. Escherichia coli bacteria were engineered to express active peptides and fed loss-of-function Caenorhabditis elegans to rescue gene activity. Using this approach, the activity of different neuropeptide genes with varying lengths and functions: trh-1, ins-6, and pdf-1 were rescued. While some peptides are functionally redundant, others exhibited unique and previously uncharacterized functions. The mechanism of peptide uptake is reminiscent of RNA interference, suggesting convergent mechanisms of gene regulation in organisms. The rescue-by-feeding paradigm provides a high-throughput screening strategy to elucidate the functional landscape of neuropeptide genes regulating different behavioral and physiological processes. Neuropeptide rescue-by feeding, expands on RNA interference (RNAi) feeding paradigms, which have been successfully used to test gene function, in which a plasmid encoding the RNA of interest is driven by isopropyl-β-D-thiogalactoside (IPTG)-induction.

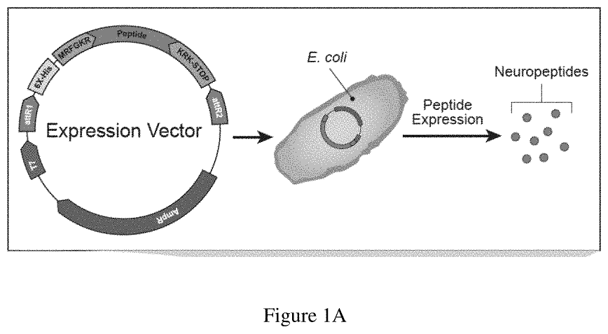

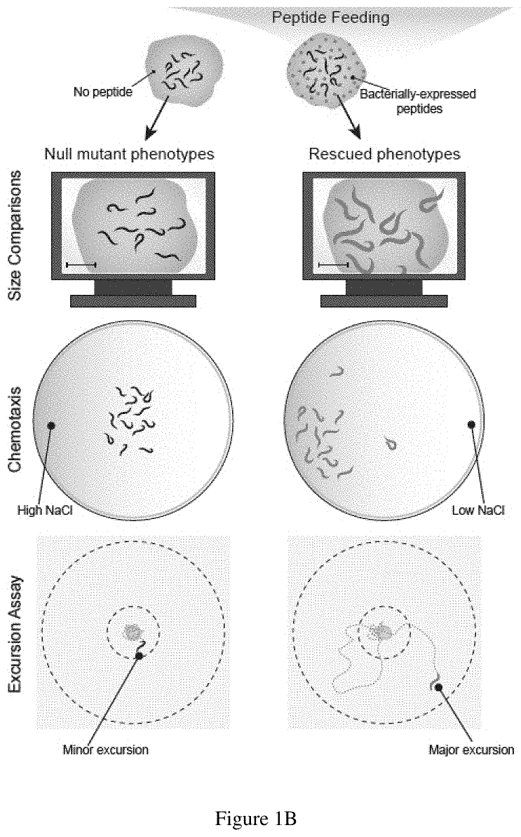

RNAi employs paired T7 promoters facing one another to produce double-stranded RNA, the methods described herein uses one T7 promoter to generate mRNA encoding the peptide of interest. The use of Gateway Cloning to develop expression vectors allows for the development of high-throughput experiments targeting individual peptides, or combinations thereof (FIG. 1A). Additionally, this technique is readily accessible compared to traditional transgenic rescue approaches. The paradigm rescues individual, processed neuropeptides, leveraging the genetic amenability of the C. elegans food source, Escherichia coli, to circumvent the need for transgenic development and enable high-throughput rescue and elucidation of individual neuropeptide function. The examples described herein demonstrate the application of this paradigm by rescuing behaviors driven by neuropeptides synthesized from trh-1, ins-6, and pdf-1 (FIG. 1B).

Sex-specific behaviors are unique aspects of survival throughout the animal kingdom from invertebrates to humans. These behaviors include a wide range of coordinated and genetically preprogrammed social and sexual displays that ensure successful reproductive strategies, ultimately resulting in survival of the species in its natural environment. The neural circuits regulating these behavioral responses are conserved, and often shared between sexes, but dependent on social experience and physiological state. For example, the vomeronasal and main olfactory epithelium in mice are required for male aggression and mating, but in females they contribute towards receptivity and aggression. Prominent among these stimuli are mating cues. The visual displays of higher order animals are among the most apparent of the mating cues, chemical mating cues are the most ubiquitous, with entire sensory organs dedicated to pheromone sensation.

Pheromones are small-molecule signals between conspecifics that convey information on the sender's current physiological state, and potentially life stage and developmental history. The nervous system response to these stimuli is dependent on both the internal, physiological state of the animal, and external, concurrently sensed stimuli.

Nematodes communicate through a large and growing class of pheromones termed ascarosides (ascr). These small molecules convey social as well as developmental information, and the assays used to understand the roles of these cues have varied. There are multiple ascarosides found to communicate attractive behaviors, specifically in a sex-specific manner, including: ascr #1, ascr #2, ascr #3, ascr #4, and ascr #8. Unique among ascaroside structures is the presence of a p-aminobenzoate group—a folate precursor that C. elegans are unable to synthesize, and which are obtained from bacterial food sources—at the terminus of ascr #8. This pheromone has previously been shown to act as an extremely potent male attractant, being sensed via a chemosensory pathway shared with ascr #3: the male-specific CEM neurons. However, whereas ascr #3 is also sensed by over half a dozen chemosensory neurons, ascr #8 has only been shown to be sensed by the male-specific CEM.

While the CEMs offer a sex-specific mode of chemosensation for ascr #8, neuromodulators and hormones are heavily implicated in all stages of the of sex-specific and pheromone-elicited C. elegans mating behaviors. Prior to sensation of mating cues, the mate searching behavior of male C. elegans is modulated by the neuropeptide, PDF-1. This neuropeptide also controls the sexual identity of the ASJ chemosensory neurons. The mating pheromone ascr #3 is modulated by insulin signaling, while activation of ascr #3-sensing neurons also activates the NPR-1 receptor. Finally, the physical act of male sexual turning during mating is mediated by multiple FMRF amide-like peptides. This complex regulation of behaviors relies on specific neuropeptide-neuropeptide receptor (NP/NPR) modules.

Unique NP/NPR modules are known to drive specific physiological and behavioral responses in C. elegans. While DAF-2 propagates insulin-like peptide (ins) signaling, the specific peptide determines the effect. For example, ins-4 functions in learning, while ins-6 affects synapse formation. Meanwhile, avoidance of ascr #3 by hermaphrodites is mediated in part by INS-18/DAF-2 signaling—higher levels of ins-18 expression result in lower ascr #3 avoidance rates. Conversely, FMRFamide-like peptide (flp) genes, many of which encode multiple peptides, signal through a complex network in which multiple receptors sense identical peptides, and multiple FLP peptides activate the same receptors. For instance, activation of NPR-4 by FLP-18 modulates reversal length, while the sensation of the divergent FLP-4 by the same receptor contributes to food preference choice.

The examples described herein investigate the neuronal mechanisms governing the behavioral attractive response of male C. elegans to ascr #8. Males exhibit a unique behavioral tuning curve to ascr #8, preferring concentrations in the 1 pM range, no longer being attracted to higher concentrations. Given that multiple flp NP/NPR modules have been shown to play roles in setting physiological state, as well as linking sensation to physiology and behavior, Inventors here envision that peptidergic signaling is likely to play a role in the male ascr #8 behavioral response.

Previous studies of C. elegans behavioral responses to attractive social ascarosides employed a Spot Retention Assay (SRA). However, the examples described herein show that the SRA contains several drawbacks, including male-male contact and the inability to track individual animals through the course of an assay. To address these issues, a single worm attraction assay (SWAA) was developed which is a more robust assay that determines variables on a per-worm basis, and not solely at the population level. The SWAA is used in examples herein to examine the responses of him-8 males defective in flp neuropeptide genes expressed in male-specific neurons; flp-3, flp-6, flp-12, and flp-19. The examples described herein observed that flp-3 plays a role in determining the sex-specific behavioral valence: i.e., determining whether the response to ascr #8 is attractive or aversive.

Two divergent FLP-3 receptors responsible for sensing the processed neuropeptides were identified. Receptor activation studies elucidated that the previously identified flp-3-sensing G protein-coupled receptor, NPR-10, and the novel FRPR-16, are both activated by FLP-3 peptides at nanomolar affinities. Additionally, loss-of-function mutations in either receptor result in behavioral defects that parallel those observed in flp-3 mutants.

To understand the role of flp-3 more completely in mediating the ascr #8 behavioral response, a peptide rescue-by-feeding protocol was adapted. Using this method, rescue individual peptides in flp-3 mutant animals were observed and showed that a specific subset of FLP-3 peptides responsible for suppressing the avoidance differs from those responsible for driving male attraction to ascr #8.

Individual neuropeptides encoded by the flp-3 gene exhibit specific biological activity, by binding multiple receptors, to drive the behavioral valence to a cue in a sex-specific manner.

The methods described herein prepare bacterial rescue constructs in recombinant DNA vectors that express individual neuropeptides.

The coding sequence of neuropeptides are inserted into a Gateway Technology Vector system. DNA Oligos are ordered from, and synthesized, by third-party vendors, such as IDT Technology. Oligos are designed to include sequences that install flanking cleavage sites around the peptide sequence of interest. Individual Oligos are annealed according to IDT's protocol before inserting into the Gateway System “Donor Vector”, pDONR221. Following the “BP Reaction”, this vector is then subjected to the “LR Reaction”, in which the sequence of interest is transposed into the pDEST527 vector available from Addgene. (Plasmid #11518, provided by Dominic Esposito). Purified, final Expression Vectors are transformed into competent DH5α E. coli cells.

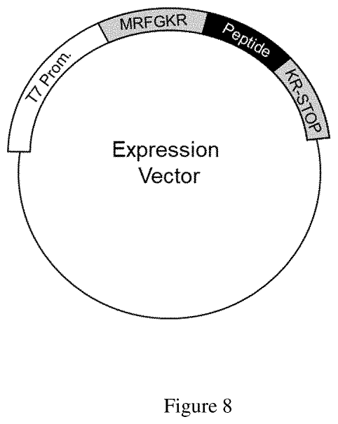

For control studies, a “scramble” vector was designed. The peptide coding sequence placed between the cleavage sites is taken from the pBluescript plasmid, for a final expression sequence of: NSKLHRGGGRSRTSGSTGSMASHARGSPGLQ (SEQ ID NO: 2) (See FIG. 8).

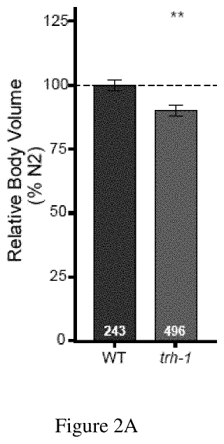

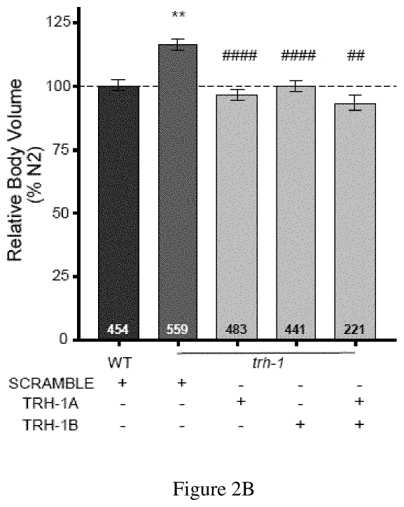

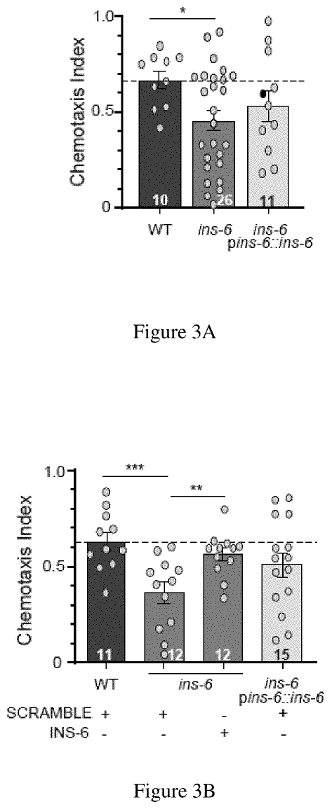

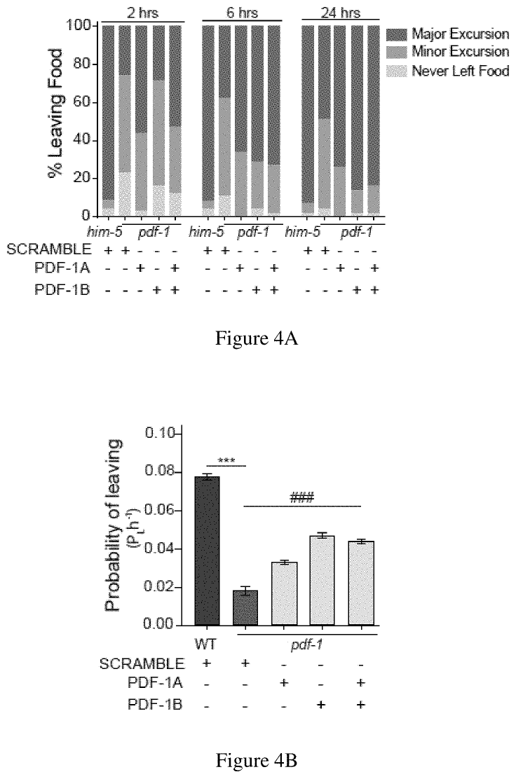

Described herein are methods of neuropeptide rescue that exploit bacterial expression to deliver individual peptides to rescue behavioral and growth-related phenotypes. While some peptides are redundant in function (FIG. 2A-FIG. 2B), others rescue phenotypes driven by large peptides (FIG. 3A-FIG. 3B), while novel functions are displayed by other peptides over long timescales (FIG. 4A-FIG. 4B). The novel technology to rescue peptide function is advantageous over transgenic studies, as it allows for functional characterization of individual peptides. This genetic tool is built off the principles of RNAi feeding techniques to supply worms with the peptide of interest through their food source as previously shown for the scorpion venom peptide mBmKTX to alter lifespan and egg-laying behavior in C. elegans. More recently, the paradigm is used to characterize the FMRFamide-like peptide gene, flp-3. This gene encodes ten different peptides and leveraged the rescue-by-feeding technology to elucidate that only two of the ten peptides encoded by the precursor are active in controlling the behavioral response of males to a mating pheromone. These examples support the assertion that feeding peptides to C. elegans via their E. coli food source is sufficient to rescue mutant phenotypes.

Based on examples described herein, it is here envisoned that the neuropeptide rescue-by-feeding strategy delivers mRNA ready for translation by the C. elegans cellular machinery, rather than supply C. elegans with fully translated and processed peptides. The plasma membrane of a cell is an intricate complex of multiple lipid and protein molecules. Small molecules with moderate polarity diffuse through the cell membrane passively, but most metabolites and short peptides require specialized membrane transporters for translocation. Given the large number of neuropeptides encoded in the genome of C. elegans, having specialized transporters for peptide transport is not feasible. The strongest piece of evidence for this statement lies in that the processed INS-6 peptide is 54 amino acids in length: the C. elegans intestine expresses peptide transporters that only uptake smaller, inactive di- and tri-amino acid peptide chains. Thus, rescue of chemotaxis behavior by INS-6 peptide feeding (FIG. 3B) suggests that the mechanism of rescue may be similar to double-stranded RNA (dsRNA) uptake, rather than peptidergic transport across the intestinal membrane. Furthermore, previous studies have shown that dsRNA exhibit transgenerational inheritance, despite degradation rates. The rescue of the exploratory behavior of pdf-1 lof males even in the 24-hour timescale (FIG. 4A-FIG. 4B) suggest that the feeding paradigm exploits similar mechanisms, allowing peptide-encoding mRNAs to remain present throughout the assay. If peptides were taken up, their degradation rates would likely impede rescue efficiency at later timepoints unlike what is observed in the examples suggesting that the mechanism is similar to double-stranded RNA (dsRNA) feeding, with mRNA uptake driving rescue rather than peptide uptake.

Feeding E. coli DH5α expressing peptides results in altered phenotypes compared to E. coli OP50 (FIG. 2A-FIG. 2B, and FIG. 3A-FIG. 3B). Given the differences in nutrient composition of the different E. coli strains and worms preferring more nutritious bacteria, expressing peptides in HB101 or HB115 strains offer viable alternatives. Understanding neuropeptide function is essential both from a perspective of regulation of neuronal (synaptic and non-synaptic) channels of communication, and form a global view of general neuronal functional assignments throughout the brain. Revealing how a particular neuropeptide acts both at the cellular and subcellular peptide receptor level is critical link in understanding the role of neuromodulation of circuits. An enhanced experimental pipeline to investigate peptide function will enable progress toward answering how neural circuit activity within the network in its different states and identification are modulated by neuropeptides, resulting in flexible decision-making during behaviors.

### Rescue-by-Feeding Protocol

SCRAMBLE control or peptide (rescue) constructs are grown overnight in LB media containing 50 μg/μL ampicillin at 37° C. and diluted to an OD600 of 1.0 prior to seeding on NGM plates containing 50 μg/μL ampicillin and 1 mM IPTG. A 75 μL lawn is left to dry and grow overnight at room temperature before 3 larval-stage 4 (L4) C. elegans are placed on the plates. Animals selected for testing are isolated onto plates also seeded with the same peptide on which they had been cultured.

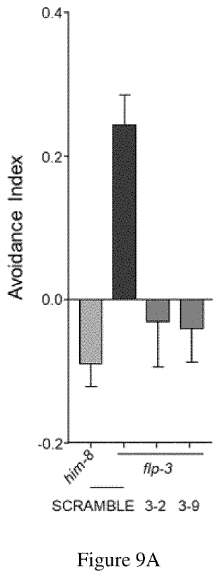

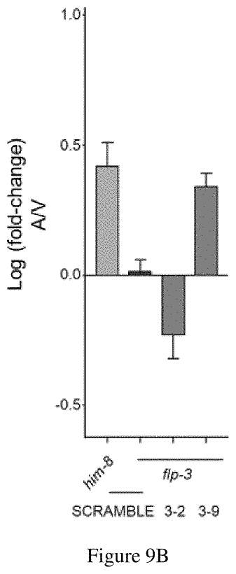

FLP-3 mutant males are no longer attracted to a mating pheromone, but rescue by two specific FLP-3 peptides (FLP-3-2 and FLP-3-9) rescue the behavior. Both FLP-3-2 and FLP-39 suppress the avoidance phenotype observed in mutants (FIG. 9A), while FLP-3-9 also rescues the attraction behavior (FIG. 9B).

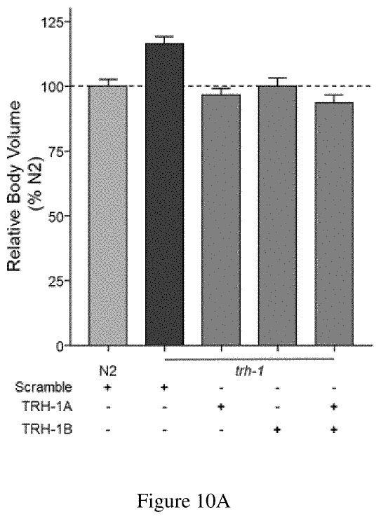

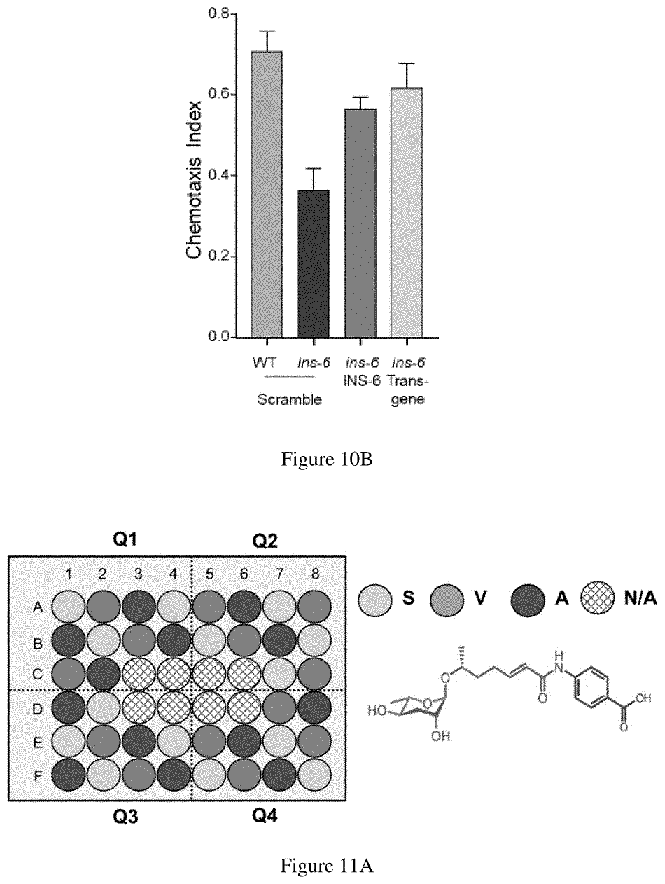

TRH-1 mutants are defective in their size during growth and development. Rescue by either TRH-1A or TRH-1B can rescue this growth defect, measured as “Relative % Body Volume”. (FIG. 10A). Meanwhile, INS-6 mutants are defective in their attraction towards NaCl (sodium chloride), measured as a “Chemotaxis Index”. Feeding of INS-6 to mutant animals restores this chemotaxis defect (FIG. 10B).

Based on the findings of INS-6 rescue, the feeding-by-rescue paradigm relies on mRNA uptake, and not peptide uptake. Studies for C. elegans digestive biology have shown that proteins are cut into small “chunks” prior to absorption across the intestinal wall. Given the size of the INS-6 precursor (112 amino acids), and even the size of the processed peptide (54 aa), it is extremely unlikely that INS-6 would be absorbed intact. Given the efficiency at which dsRNA is taken up by C. elegans in RNA-interference feeding techniques, it is here envisioned that the methods described herein exploit a similar pathway, with C. elegans taking up INS-6 mRNA directly.

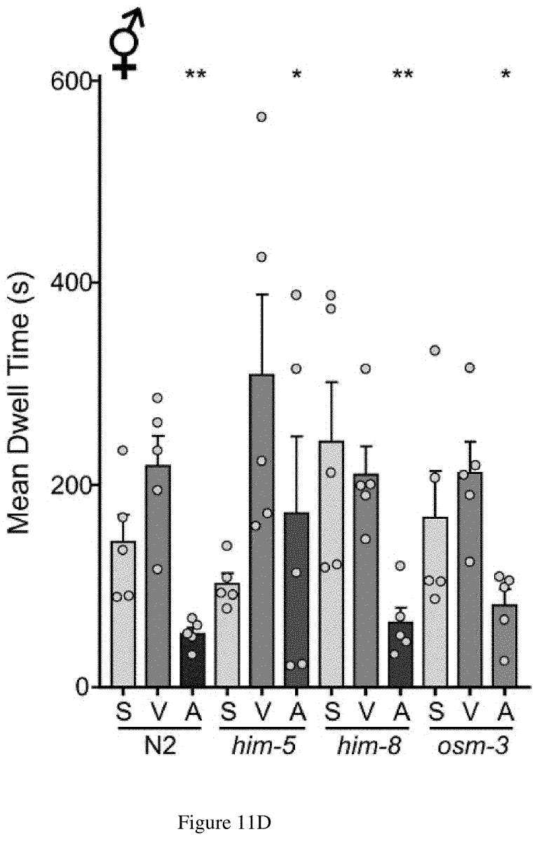

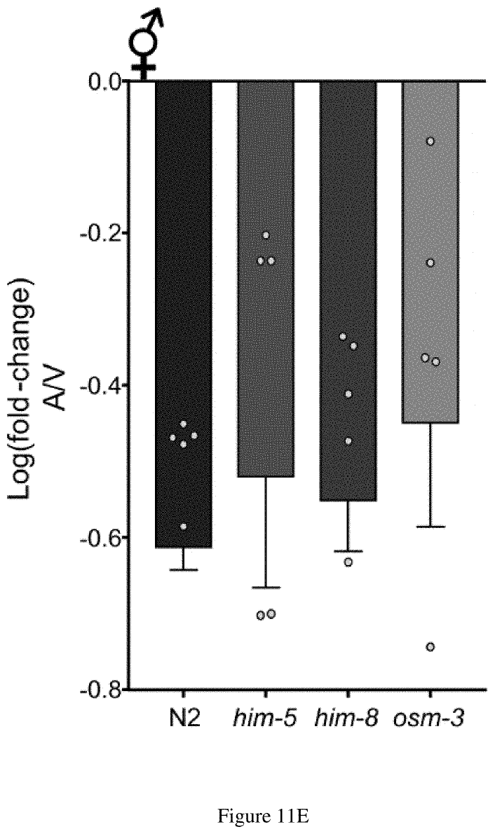

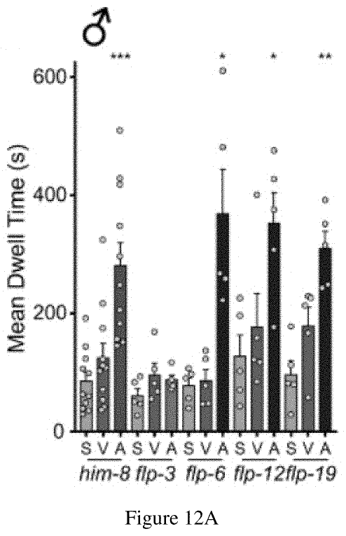

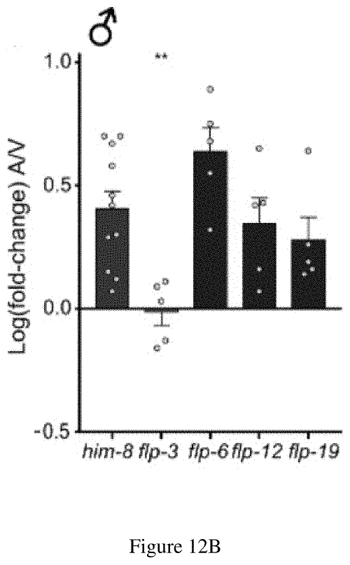

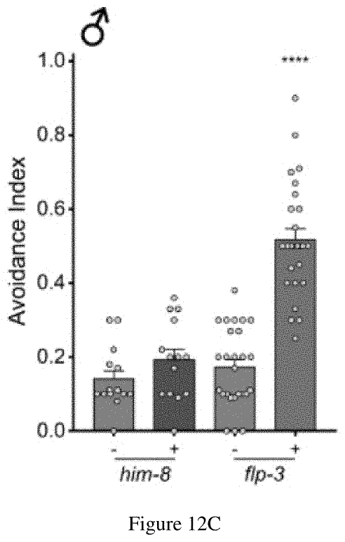

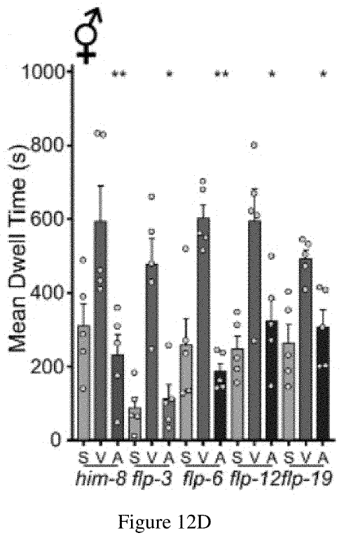

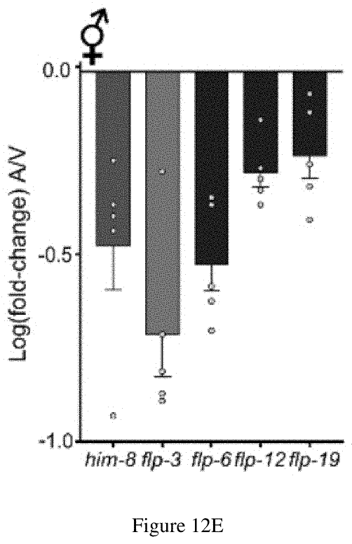

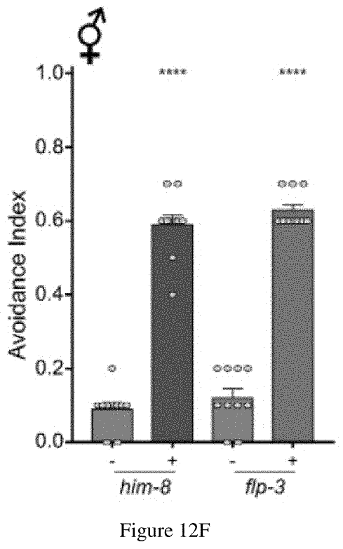

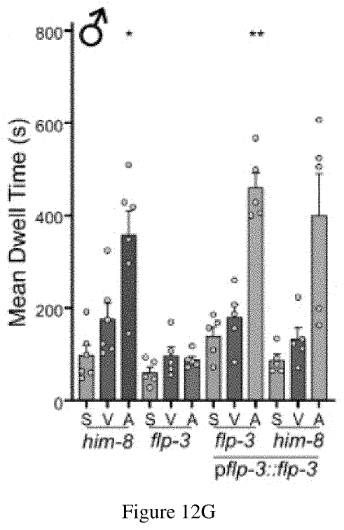

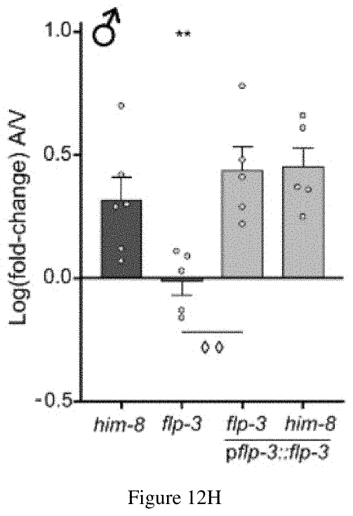

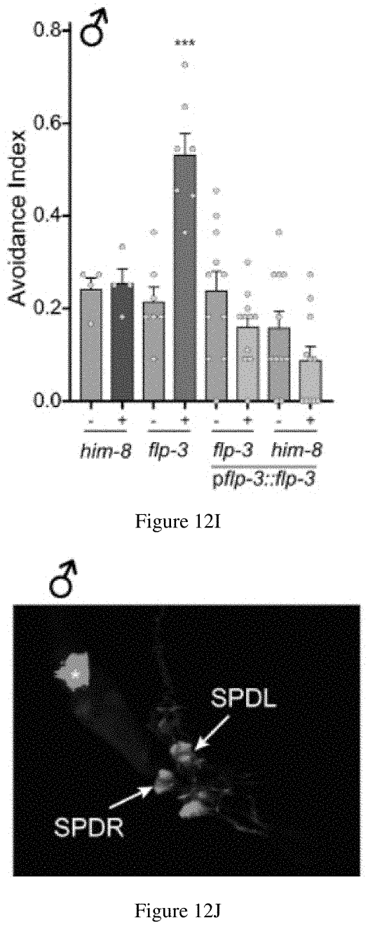

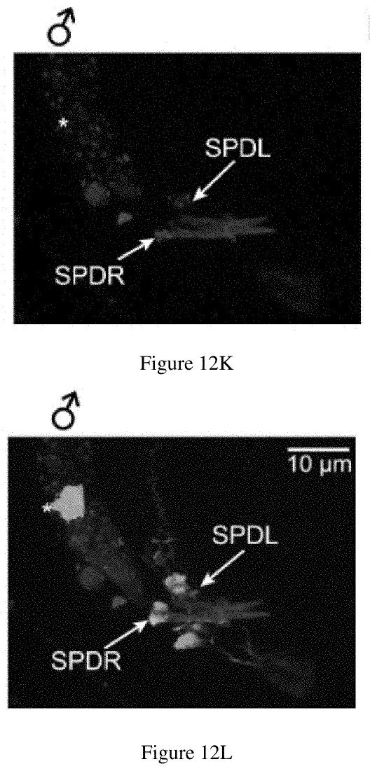

The examples described herein reveal a complex mechanism regulating the sex-specific behavioral response to a pheromone guided through the interaction of at least two peptides encoded by a single-neuropeptide precursor gene and two divergent GPCRs. The examples show that two distinct NP/NPR modules driven by the single-neuropeptide gene flp-3 serve to drive sex-specific attraction to the male-attracting pheromone, ascr #8 (FIG. 12A-FIG. 12L).

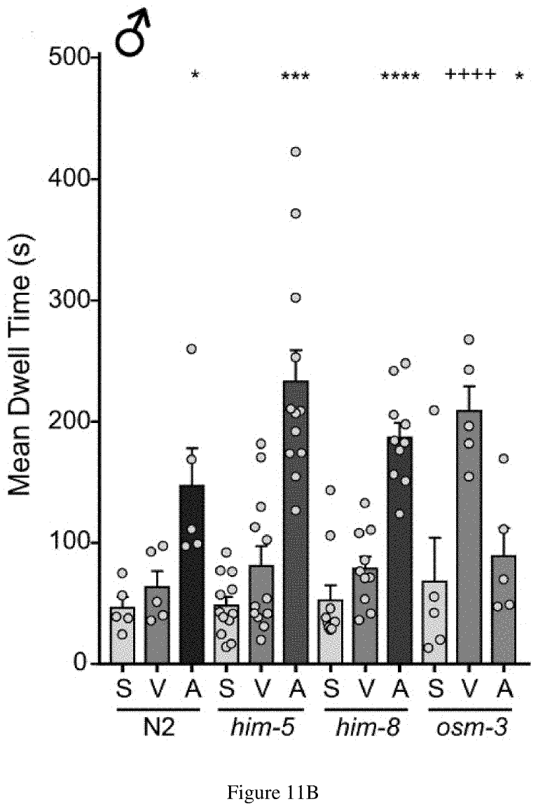

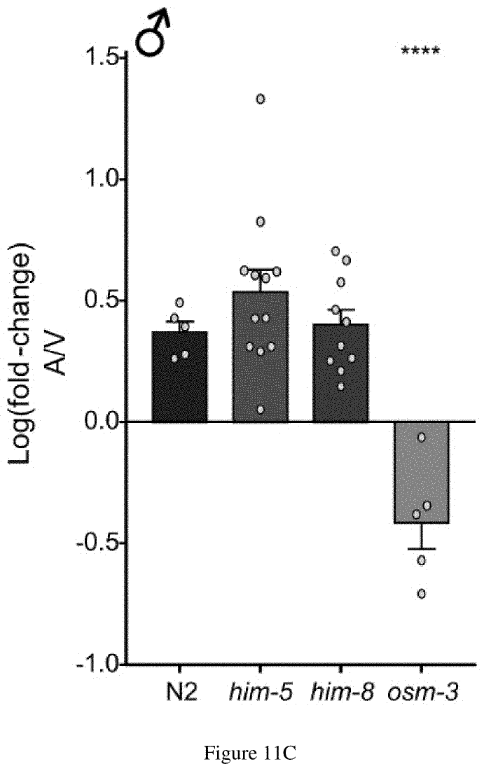

A novel single worm behavioral assay (SWAA) was developed and validated, which confirmed that male C. elegans are indeed attracted to different pheromone cues (FIG. 11A-FIG. 11E). Previous attraction assays, such as Spot retention assay (SRA), resulted in attraction values that were skewed due to male-male contact. The SWAA overcomes those caveats as it measures the attractive properties of individual animals in each spot (FIG. 11A-FIG. 11E). It also helps calculate the percentage attractiveness of the cue adding another parameter to measure the robustness of the cue. The SWAA suggests that both him-5 and him-8 males are equally attracted to ascr #8 (FIG. 11A-FIG. 11E). This addresses one of the caveats of the SRA results, wherein him-5 and him-8 males responded differentially to ascr #8, with him-8 males being significantly more attracted than their him-5 counterparts. This may be due to a high degree of male-male contact within the ascr #8 spot, but not the vehicle spot in SRA. It was additionally confirmed that another previously described male attractant ascaroside, acsr #3, maintains its ability to attract males in the SWAA. The examples demonstrate that hermaphrodites, which have previously been shown to leave food rarely in comparison to males, overcome spatial control effects reminiscent of ‘edge effects’—wherein animals spend more time along the edge rather than in the throughout the well—and instead spend more time the vehicle control more often (FIG. 11D). It was observed that this dwell time is dramatically reduced in the presence of the pheromone, supporting the findings that hermaphrodites avoid ascr #8 (FIG. 12F).

Peptidergic modulation of neural circuits as long been hypothesized as complex, and here is elucidated the recruitment of two neuropeptide/neuropeptide-receptor (NP/NPR) modules, FLP-3/NPR-10 and FLP-3/FRPR-16, that both serve to regulate the nervous system in sensing and respond to asr #8 in a sex-specific manner. The results elucidate that not all peptides encoded by the FLP-3 propeptide are involved in the regulation of the sex-specific circuit, but rather a subset function through two unique NP/NPR modules to drive the behavioral response. This suggests that the complexity of the expansive class of FMRFamide-related peptide (FLP) genes in C. elegans, of which there are 31 genes encoding over 70 unique peptides, function through an even greater number of NP/NPR modules to drive specific behavioral or physiological states. FLPs have been identified as regulators of a variety of behavioral and sensory mechanisms, including locomotion, egg-laying, gas sensing, sleep, and mating. The examples herein show that flp-3 functions to coordinate ascr #8 sensation with attractive behavior.

Previous studies have linked the entirety of the gene to a receptor based on binding studies and full transgenic rescue. The examples described herein employ a rescue-by-feeding assay, following the design of RNAi feeding protocols, to rescue individual peptides. While “feeding” of peptides through soaking is a valid approach, there are many constraints on such approaches, the most prominent being the ability to acquire purified peptides. Using the rescue-by-feeding approach, access to the peptide is provided to the worms directly through their food source. This approach enables new avenues for characterizing the roles of the other neuropeptides in mediating diverse cellular and organismal processes.

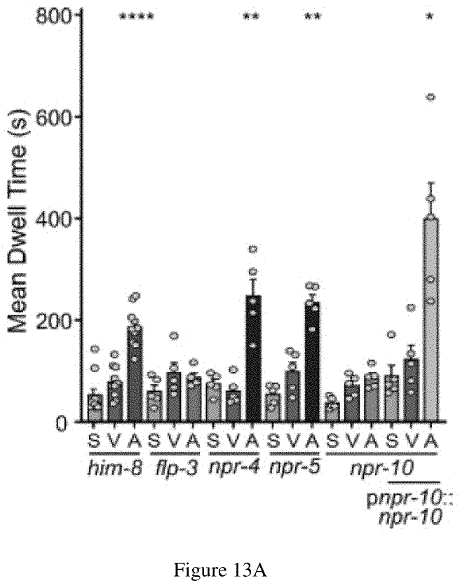

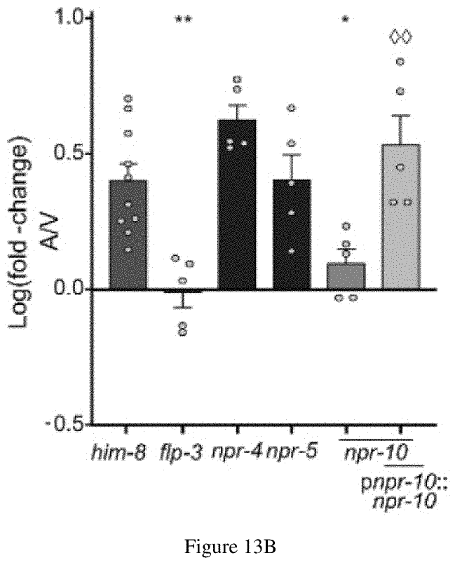

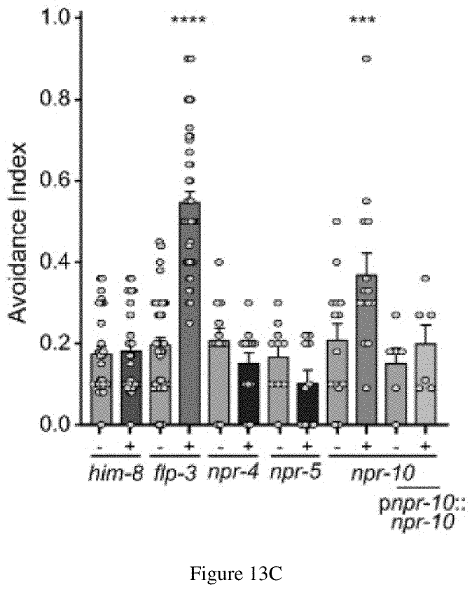

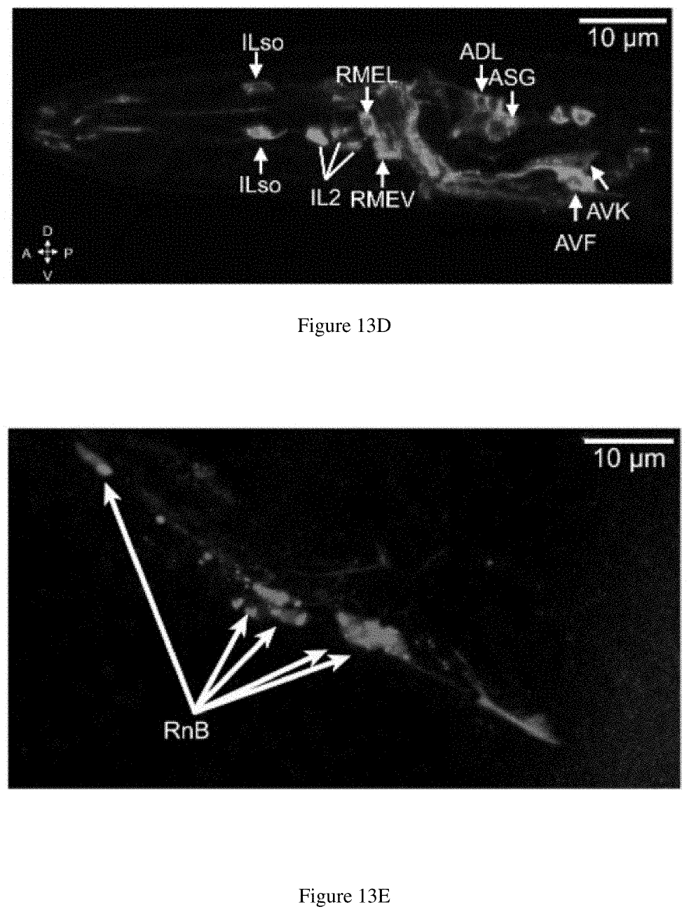

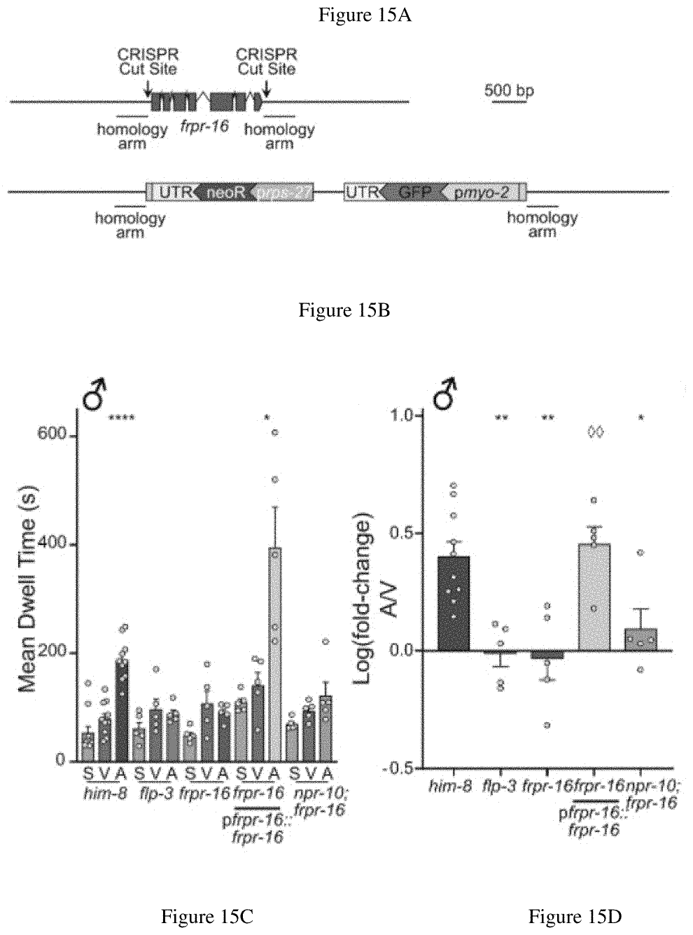

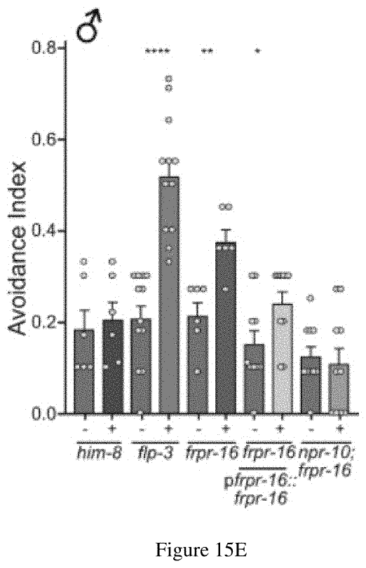

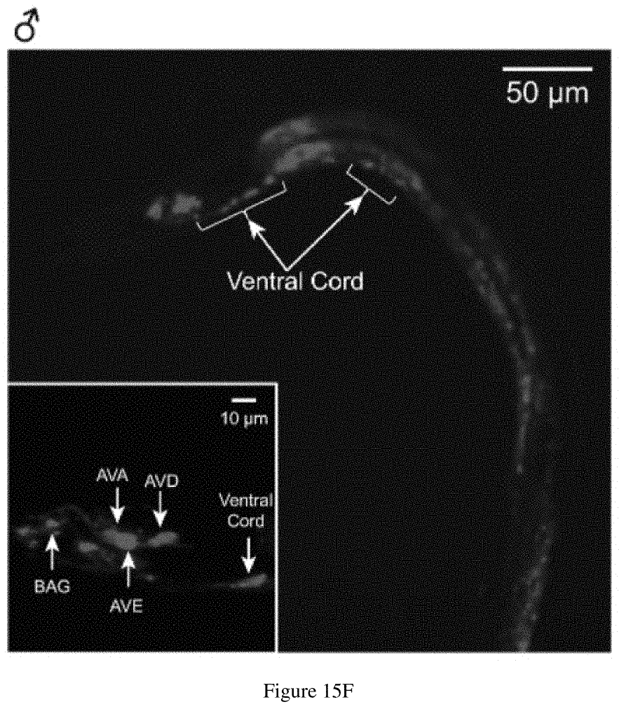

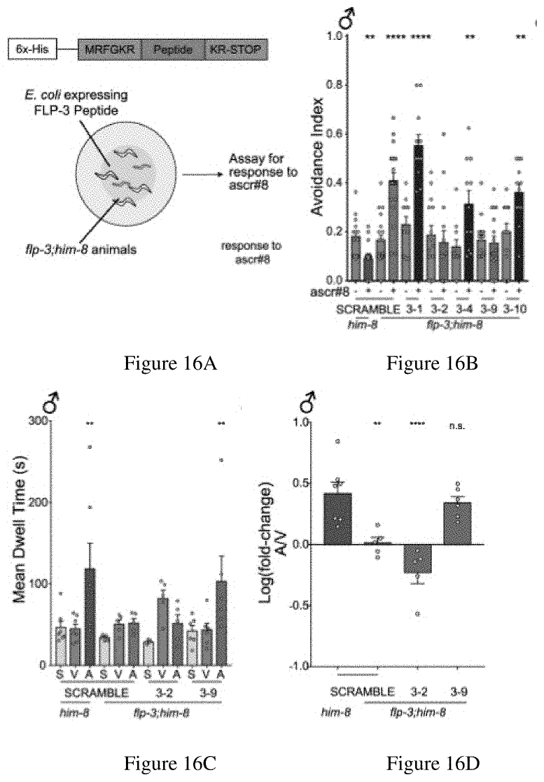

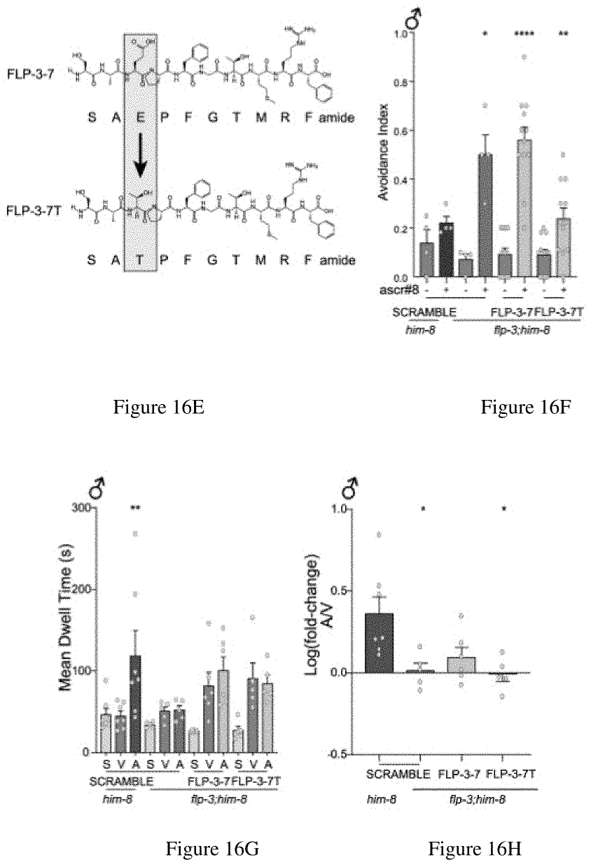

Combining biochemical receptor activation studies with behavioral rescue-by-feeding assays, the examples described herein have been successful in elucidating discrete neuropeptide signaling modalities within the complex FLP-3 signaling system. The involvement of two evolutionarily divergent receptors in sensing specific FLP-3 neuropeptides suggests that the sex-specific behavioral response to ascr #8 module is a result of the activity of two distinct NP/NPR modules that mediate both attractive and repulsive properties of the small molecule. Two GPCRs respond to FLP-3 peptides to function in the behavioral response to ascr #8: the previously identified NPR-10, and the novel FRPR-16 (FIG. 13A-FIG. 13E, FIG. 15A-FIG. 15F). Both exhibit high potencies for multiple FLP-3 peptides, although our single-peptide rescues have shown that FLP-3-2 and FLP-3-9 are required for the wild-type response to ascr #8 (FIG. 16A-FIG. 16H), while FLP-3-1, FLP-3-4, and FLP-3-10 are not. As such, multiple NP/NPR modules are implicated in the ascr #8 behavioral response. Further studies will allow for further separation of FLP-3-2 and FLP-3-9, and how they interact with NPR-10 and FRPR-16.

Interestingly, these two high-potency FLP-3 receptors are extremely divergent in their evolutionary history. NPR-10 is most related to other “NPR” C. elegans receptors that evolved from the same family as the Drosophila melanogaster Neuropeptide F Receptor family, and exhibits predicted NPY activity. FRPR-16, however, is more closely related to the fly FMRFamide Receptor. Interestingly, while some C. elegans FRPR receptors function as FLP receptors, at least one receptor within the same evolutionary clade (DAF-37) acts as a chemosensor for pheromones. The evolutionary distance between NPR-10 and FRPR-16 suggests that these two receptors have undergone convergent evolution.

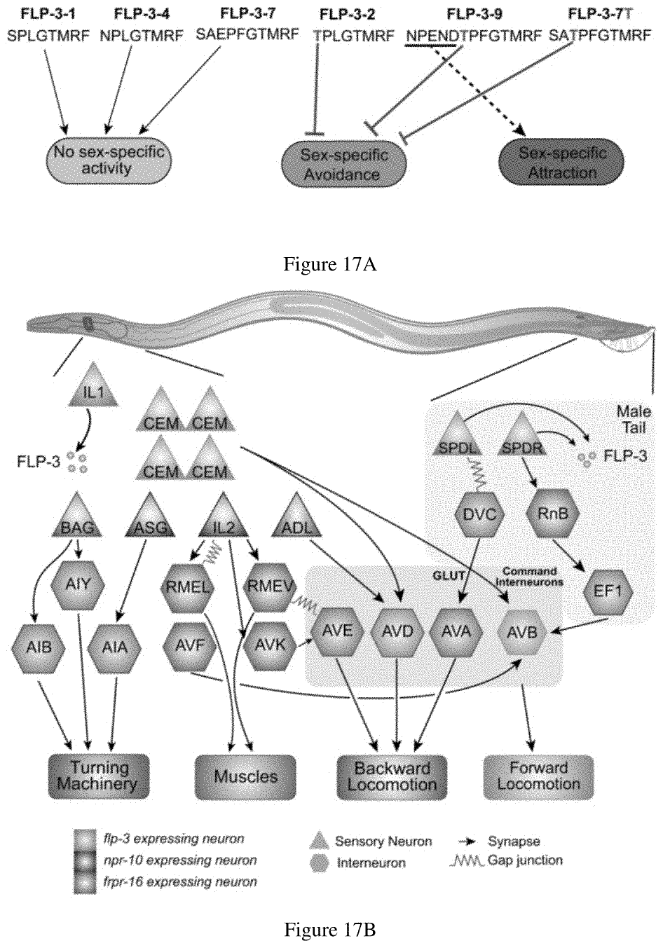

The presence of FRPR-16 in the amphid premotor interneurons responsible for backwards locomotion (FIG. 15A-FIG. 15F) suggests the FLP-3/FRPR-16 module serves to mediate reversals during ascr #8 sensation (FIG. 17B). Conversely, while FRPR-16 is confined to a small, yet biologically specific subset of neurons in the head of the animals, NPR-10 exhibits more promiscuous expression that innervates both forward and reverse locomotion circuitries (FIG. 13E-FIG. 13F). As such, while the FLP-3/FRPR-16 module specifically modulates reversals, the FLP-3/NPR-10 module may instead serve to balance both forward and backwards locomotion in response to ascr #8, allowing the animal to interrogate its surroundings more thoroughly.

The loss of both of these modules that underlies the behavioral response in npr-10;frpr-16 double mutant animals is due to ascr #8. The loss of only one module results in a skewed behavioral response, observed as aversion to ascr #8 (FIG. 13E-FIG. 13F, FIG. 15A-FIG. 15F). This aversion likely arises from the absence of amphid NPR-10 leading to aberrant regulation of turning machinery (through ASG-AIA connections) and muscle innervation (via RME neurons). In the tail, lack of NPR-10 seems to prevent EF1 activation, and thereby allowing forward locomotion to continue. Meanwhile, loss of only FRPR-16 will lead to the inability of FLP-3 to regulate backward locomotion machinery (FIG. 17B). However, the loss of both the FLP-3/NPR-10 and FLP-3/FRPR-16 modules abolishes the ability of the animal to response to ascr #8 at all, observed as neither attraction nor aversion (FIG. 15A-FIG. 15F). The absence of both receptors results in the inability of FLP-3 to suppress forward locomotion (through NPR-10) nor drive backwards locomotion (via FRPR-16) in response to acsr #8. Future studies incorporating cell-specific rescue of both NPR-10 and FRPR-16 will further elucidate this circuitry.

While the examples herein only investigated the activity of single peptides, FLP-3 is a complex gene encoding 10 discrete peptides. Not all peptides are sufficient to rescue the ascr #8 behavior on their own, they may instead serve a synergistic role to “active” peptides. The rescue-by-feeding approach makes it easy to perform combinatorial studies of peptides, allowing for the elucidation of such synergistic peptide function. However, other reasons for the lack of rescue are possible, including rapid rates of degradation of the rescue peptide RNA. Future studies should make note to carefully dissect negative results, using either peptide soaking, or comparison to in vivo expression with partial null mutants. The second course of action is discerning that FLP-3-1 and FLP-3-4 are insufficient to suppress ascr #8 avoidance in either the flp-3(ok3625) allele, or in thr rescue-by-feeding paradigm (FIG. 16A-FIG. 16H).

The findings highlight the complexity of neuromodulators regulating behavioral states in both invertebrates and vertebrates. Multiple hormonal receptors are involved in regulating the induction of A. aegyptii ecdysteroid hormone production through two different neuropeptide signaling systems: ILP3 initiates digestion of the blood meal, while OEH stimulates oocyte yolk uptake. Melanin-concentrating hormone is a neuropeptidergic hormone that promotes appetite and feeding behaviors in mice in a sex-dependent manner. Meanwhile, age-dependent changes in levels of Neuropeptide F result in the promotion of survival-benefiting appetitive memory in Drosophila, concurrent with the impairment of memories associated with insufficient survival benefits.

Sex-specific behaviors arise from processing specialized olfactory cues emitted by their conspecifics. Odor processing within each sex is mediated by flexible neural circuits and neuromodulation enables neural networks to adapt behaviors under fluctuating external and internal environmental states. So how is this adaptability achieved? The examples demonstrate that specific peptides encoded by a single-neuropeptide gene, activate evolutionarily divergent receptors resulting in fine-tuned sex-specific behavioral responses to small-molecule pheromones. These NP/NPR modules are expressed within specific neurons of the nervous system of the two sexes, mediating overlapping behavioral outputs, by simultaneously suppressing an avoidance response and driving an attractive response (FIG. 17B). The CEM neurons, which act as a primary site of ascr #8 sensation, synapse onto both forward (AVB) and backward (AVD) locomotory neurons, the activity of which are modulated by these NP/NPR modules both indirectly and directly, respectively (FIG. 17B). These findings highlight the complexity of peptidergic modulation of the nervous system, wherein individual peptides either from a single gene or multiple genes modulate opposing behaviors, through multiple NP/NPR modules. Using the rescue-by-feeding paradigm, the function of discrete peptides can be unraveled, enhancing the understanding of pathways of extra synaptic information flow in the complex functional connectome.

A portion of the embodiments described herein were published in BioRxiv as “Non-Transgenic Functional Rescue of Neuropeptides” by co-authors Elizabeth M. DiLoreto, Douglas K. Reilly, and Jagan Srinivasan. Another portion of the embodiments described herein were published in Commun Biol 4, 1018 (2021) as “Distinct neuropeptide-receptor modules regulate a sex-specific behavioral response to a pheromone” by co-authors Douglas K. Reilly, Emily J. McGlame, Elke Vandewyer, Annalise N. Robidoux, Caroline S. Muirhead, Haylea T. Northcott, William Joyce, Mark J. Alkema, Robert J. Gegear, Isabel Beets, and Jagan Srinivasan. These publications are hereby incorporated by reference herein in its entirety including any supplementary data.

The inventions described herein are the most practical methods. It is recognized, however, that departures may be made within the scope of the invention and that modifications will occur to a person skilled in the art. With respect to the above description then, it is to be realized that the optimum dimensional relationships for the parts of the invention, to include variations in size, materials, shape, form, function, steps, and manner of operation, assembly and use, would be apparent to one skilled in the art, and all equivalent relationships to those illustrated in the drawings and described in the specification are intended to be encompassed by the present inventions.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention. Such equivalents are within the scope of the present invention and claims. The contents of all references including issued patents and published patent applications cited in this application are hereby incorporated by reference.

The invention now having been fully described, it is further exemplified by the following examples and claims.

### Example 1: Functional Redundancy of Thyrotropin-Releasing Hormone (TRH)-Like Peptides in C. elegans

Recent work has revealed that C. elegans express homologs of mammalian thyrotropin releasing hormone (TRH). Expression of the nematode gene, trh-1, in the pharyngeal motor neurons M4 and M5 results in the production of a TRH-like neuropeptide precursor that is processed into two matured peptides: TRH-1A (GRELF-NH₂)(SEQ ID NO: 3), and TRH-1B (ANELF-NH₂) (SEQ ID NO: 4). Like FLP and other NLP peptides, these peptides are also flanked by di/tribasic residues, allowing them to be processed by EGL-3.

In mammals, TRH is essential for proper metabolism and growth, and can even induce metamorphosis in certain amphibians. The initial characterization of trh-1 and the cognate receptor gene, trhr-1, revealed a similar role within the C. elegans nervous system. Animals expressing a trh-1 pro-peptide that is truncated prior to peptide translation due to an 8-bp indel-frameshift are significantly shorter and thinner than their wild-type counterparts, resulting in a relative body volume defect (FIG. 1B, Size Comparisons), 48-hours after larval stage 1 (L1) arrest (Mann-Whitney test, p=0.0012) (FIG. 2A). In-vitro studies involving biochemical activation of the TRHR-1 receptor by either peptide (TRH-1A or TRH-1B) suggests functional redundancy of these peptides in rescuing body volume defect in trh-1 animals.

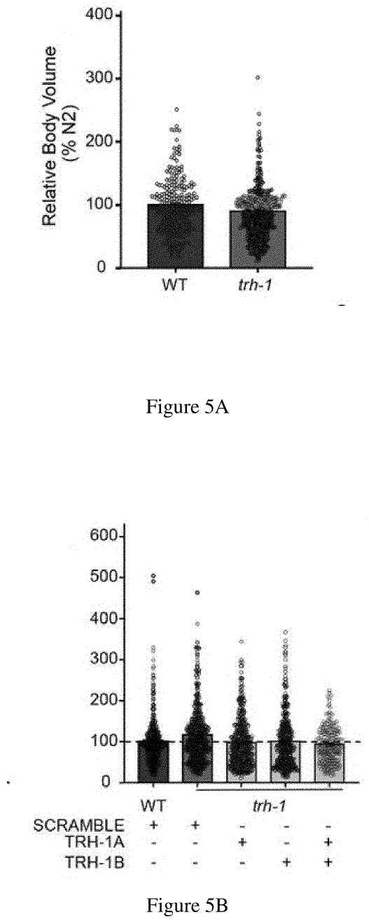

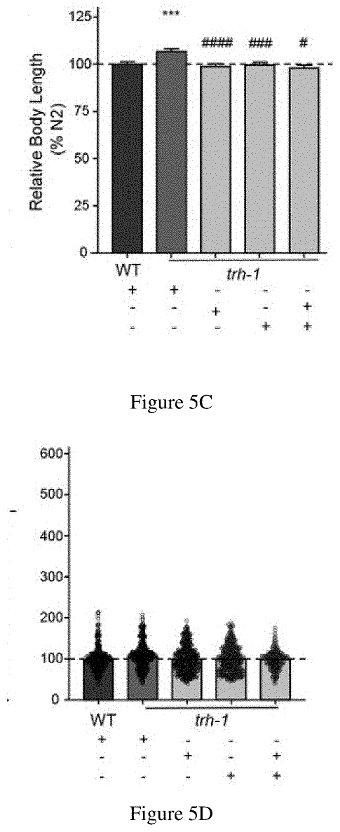

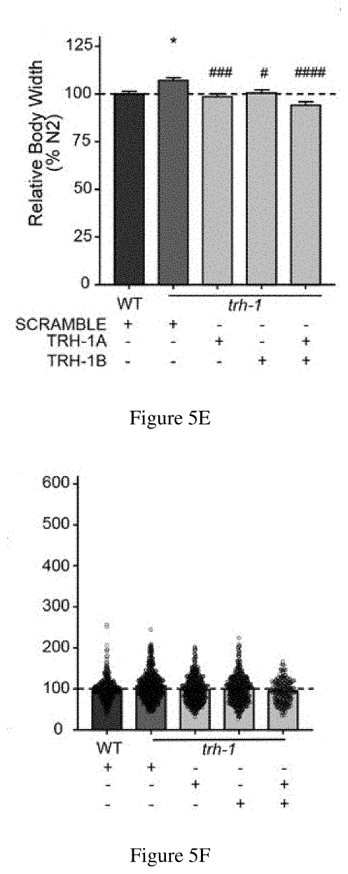

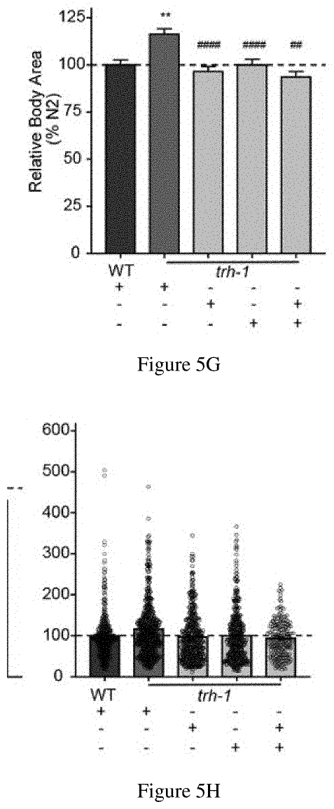

Both TRH-1 peptides; TRH-1A and TRH-1B were cloned in the expression vector as described in FIG. 1. The trh-1 loss-of-function (lof) mutant animals fed scramble peptide (SCRAMBLE, NSKLHRGGGRSRTSGSTGSMASHARGSPGLQ-NH2) (SEQ ID NO: 2) expressed in E. coli DH5α cells experience a significant increase in their body volume compared to wild-type animals (Kruskal-Wallis followed by Dunn's multiple comparison test, p=0.0015) (FIG. 2B). However, feeding trh-1 lof animals with E. coli bacteria expressing either TRH-1A, or TRH-1B (trh-1 lof versus TRH-1A or TRH-1B fed, p<0.0001) or a combination of both TRH-1A and TRH-1B peptides resulted in a complete restoration of wild-type body volume (trh-1 lof versus TRH-1A+B fed, p=0.0019; wild-type versus TRH-1A+B fed, p>0.9999) (FIG. 2B, FIG. 5A-FIG. 5H for length, width and area). This suggests that feeding bacteria expressing TRH-1A or TRH-1B, or a combination of the two peptides, to trh-1 mutant animals results in rescue of body volume, suggesting that they are functionally redundant. While transgenic C. elegans expressing the full-length trh-1 gene under its endogenous promoter restores wild-type body volume, the role of individual peptides (TRH-1A and TRH-1B) in regulating this phenotype was not addressed. The rescue-by-feeding strategy offers an easy, high-throughput in vivo biological confirmation of function.

It was observed that the quality of food determines the body morphology phenotype in trh-1 mutant worms (FIG. 2A, FIG. 2B). The trh-1 mutant worms when fed on the standard food source, E. coli OP50, displayed reduced body volume (FIG. 2A). However, trh-1 lof worms reared on E. coli DH5α cells exhibited increased body volume compared to wildtype animals raised under similar conditions (FIG. 2B). In addition, it was observed that other body morphology characteristics such as relative body length (FIG. 5C-FIG. 5D), width (FIG. 5E-FIG. 5F), and area (FIG. 5G-FIG. 5H) are different in worms fed with E. coli DH5α compared to E. coli OP50 fed animals. E. coli OP50 strain, an uracil auxotroph is the most commonly used laboratory bacterial food source as it grows in thin lawns which allow easier visualization of worms. However, studies have shown that C. elegans prefer other more nutritious bacteria such as HB101 or Comamonas sp. for its nutrition. It is here hypothesized that the reduced body volume defect in trh-1 lof worms reared on E. coli OP50 could be a result of an inefficiency of nutrient absorption due to a difference in the nutrient composition of the two E. coli strains DH5α and OP50. The trh-1 mutant worms reared on E. coli HB101 exhibit no defect in relative body volume in compared to E. coli OP50 fed animals.

### Example 2: Feeding of INS-6 Peptides Rescues Chemotaxis Defects Exhibited by ins-6 Mutants

Insulin and insulin-like peptides serve signaling functions in Drosophila and C. elegans homologous to the human insulin-like growth factor (IGF), which regulates FOXO activity. In C. elegans, ins-6 encodes only one processed INS-6 peptide (VPAPGETRACGRKLISLVMAVCGDLCNPQEGKDIATECCGNQCSDDYIRSACCP-NH2) (SEQ ID NO: 5), though the gene was originally postulated to encode two putative proteins. The ins-6 functions in dauer formation and sensory modulation of large fluctuations in salt concentration, with loss of ins-6 causing dysfunction of NaCl attraction (FIG. 1B, Chemotaxis).

While wild-type worms were attracted to high concentrations of salt (750 mM), there was a significant decrease in attraction in ins-6 mutant animals, as measured by a Chemotaxis Index (CI) (Paired t-test with samples of equal variance, p=0.0248) (FIG. 3A). Salt attraction was partially rescued with genetic reintroduction of ins-6 under its endogenous promotor (FIG. 3A). Similarly, the CI of ins-6 lof animals fed scramble peptide was significantly different from the wild-type animals fed scramble (FIG. 3B). Feeding of E. coli expressing INS-6 peptide to ins-6 lof animals resulted in a complete restoration of attraction to high salt concentration (ANOVA, followed by Bonferroni's Correction, ins-6 lof fed scramble versus INS-6 fed, p=0.0101) (FIG. 3B).

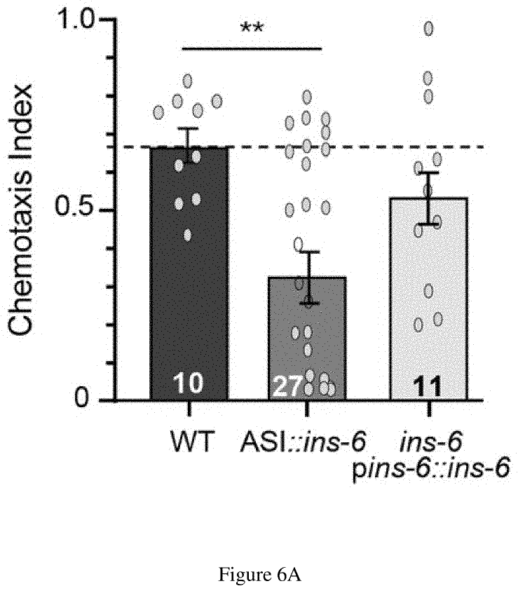

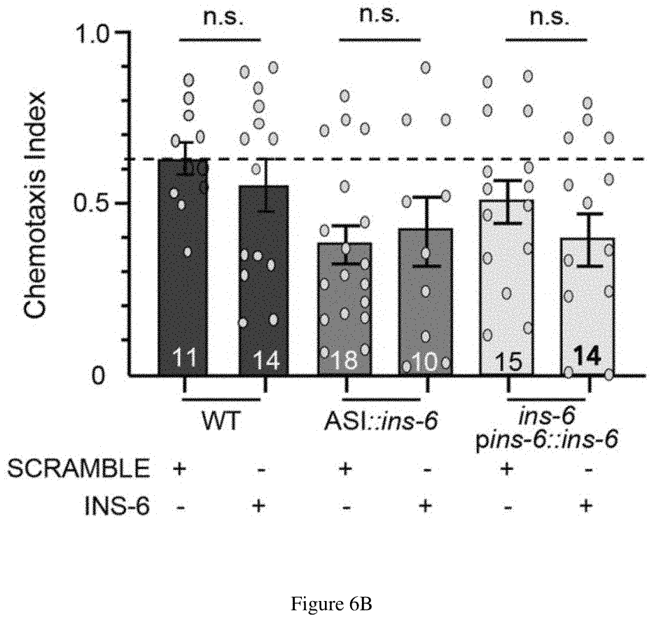

Overexpression of INS-6 did not increase chemotaxis towards high salt, as wild-type animals fed INS-6, along with a full ins-6 genetic rescue, exhibited slight defects in CI towards high salt (ANOVA, followed by Bonferroni's Correction, wild-type versus fed, p=0.08071; wild-type versus transgenic overexpression, p=0.2403) (FIG. 6B). Partial genetic rescue of ins-6 limited to the ASI sensory neuron (ins-6;ASI::ins-6) was sufficient to rescue neurophysiological function of AWC^{ON} NaCl sensation, though it was not sufficient for rescuing behavioral attraction to salt (ANOVA, followed by Bonferroni's Correction, p=0.2136) (FIG. 6B).

### Example 3: Differential Functional Activity of Peptides Encoded by Pigment Dispersing Factor (pdf)-1

Wild-type males display a characteristic exploratory behavior when left on a lawn of food, as well-fed males leave food in search of mates. The pigment dispersing factor pdf-1 neuropeptide plays a significant role in male mate-searching behavior, balancing the neural circuits controlling two predominant male interests: finding food and finding mates. Like trh-1, the pdf-1 precursor encodes two neuropeptides:

PDF-1A
(SEQ ID NO: 6)
(SNAELINGLIGMDLGKLSAVG-NH2)
and
PDF-1B
(SEQ ID NO: 7)
(SNAELINGLLSMNLNKLSGAG-NH2).

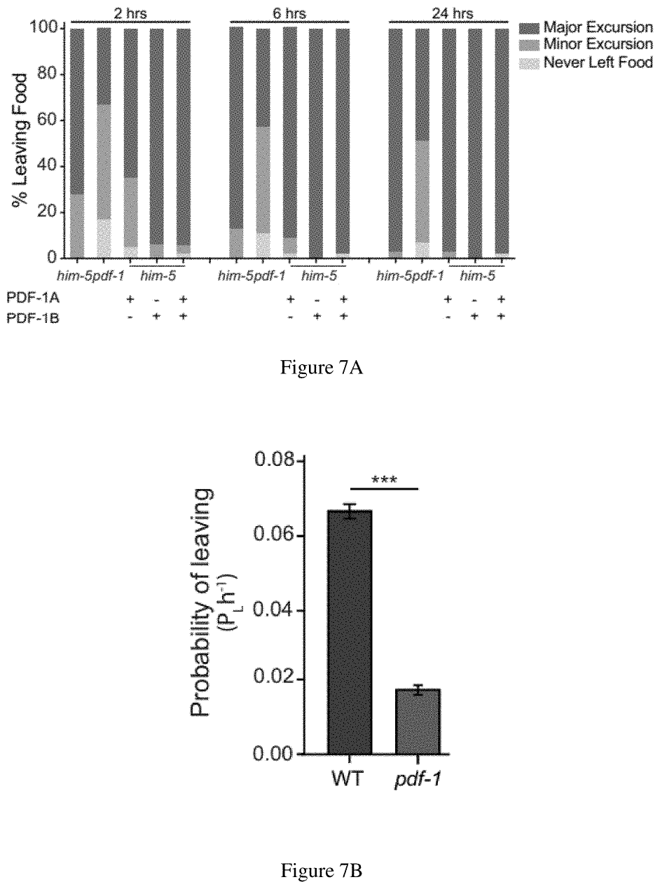

We quantified the behavioral activity of pdf-1 lof males using a food-leaving behavior as previously described. Individuals were placed on a small food spot, and track patterns were scored at three different time points (2, 6, 24 hrs) (FIG. 4A). “Never left food” indicate the absence of tracks outside the food spot. “Minor excursion” indicates that tracks were observed not beyond 1 cm from the food. “Major excursion” indicates the presence of tracks past the 1 cm boundary (FIG. 1B, Excursion Assay). The food-leaving behavior was quantified as a measure of mate-searching behavior. In this behavior well-fed males left food in search of mates and the data is represented as a Probability of Leaving (P_{L}) (FIG. 7A).

The pdf-1 lof males did not leave food as readily as wild-type worms suggesting that these worms do not display exploratory behavior, as previously described (Paired t-test with samples of equal variance, p<0.0001) (FIG. 7A and FIG. 7B). When fed either PDF-1 peptide (or a 1:1 combination of both), pdf-1 lof males had a higher P_{L} Compared to scramble fed pdf-1 lof males (ANOVA, followed by Bonferroni's Correction, p<0.0001) (FIG. 4B). However, PDF-1B and a 1:1 ratio of both peptides resulted in a significantly different P_{L} from pdf-1 males fed PDF-1A alone (ANOVA, followed by Bonferroni's Correction, p<0.0001). Rescue-by-feeding resulted in a partial rescue: while the P_{L} of peptide-fed males were significantly higher than pdf-1 lof males fed scramble, they were still significantly lower than wild-type males fed scramble peptide (ANOVA, followed by Bonferroni's Correction, p<0.0001) (FIG. 4B).

### Example 4: Spot Retention Assay Vs. Single Worm Assay

The spot retention assay was adapted to allow for better characterization of individual worm behavior and robust interrogation of attraction to small molecules. The attractiveness of 1 μM ascr #8 (FIG. 11A) were compared across multiple strains of C. elegans (the wild-type N2 strain, the high incidence of male him-5 and him-8 strains, and the chemosensory cilia defective osm-3), using the novel behavioral assay, the single worm attraction assay (SWAA) (FIG. 11A). In this assay, individual animals were placed directly into the spot of the ascaroside cue (A) while simultaneously removing any potential of male-male contact. A spatial control was included throughout the assay plate to allow investigating any innate differences in the number of visits to the well center, or the time spent therein, of which it was observed to have only one strain to date with differences in male dwell time (FIG. 1A). Likewise, a vehicle control (V) is also included, to account of any changes in dwell time that are driven by the components of the vehicle.

Male C. elegans exhibited a significant increase in the amount of time spent within ascr #8 spot compared to the vehicle control, in all wild-type and him male strains tested (FIG. 11B and FIG. 11C). These results confirm the attraction behavior of wild-type and him males observed with SRA. To measure the attraction behavior of the ascaroside, the normalized increase in dwell time was computed and calculated as log(fold-change) [i.e., ascaroside dwell time over vehicle dwell time]. Using this log(fold-change) metric, it was observed that the increase in attraction to ascr #8 is consistent across N2, him-5, and him-8 male strains (FIG. 11C). This metric enabled a direct comparison between strains and conditions while accounting for baseline variability in vehicle dwell times.

Given the setup of the SWAA assay, the number of visits per-worm under different genetic backgrounds and conditions can be measured. This assay additionally helps calculate the percentage of attractive visits to the cue. The results suggest no difference in the attractive properties to ascr #8 between any of the wild-type strains. Setting an “attractive visit” as any visit longer than two standard deviations above the mean vehicle dwell time, we show that males are indeed attracted to the cue itself, and not the male-male contact. The results suggest that it is in fact a minority of animals (30-45%) that exhibit attractive visits to the cue. This rate of behavioral attraction to ascr #8 is consistent with calcium imaging experiments wherein ascr #8 exposure elicits similar rates of calcium transients in the CEM neurons.

Unlike the SRA, wherein hermaphrodites did not exhibit any difference in dwell time between vehicle and ascr #8, the SWAA revealed that hermaphrodites from all strains consistently spent significantly less time in ascr #8 than the vehicle, with no difference between the spatial and vehicle control dwell times (FIG. 12D, FIG. 12E). Hermaphrodites also visited the ascaroside cue less than they did vehicle or spatial control well centers and exhibited little-to-no attractive visits.

Together, these data validate the SWAA as a robust assay for the measurement of the attractiveness of a small-molecule cue on a single animal basis in both sexes. In addition, it provides data on visit count and the percent of attractive visits that was previously impossible utilizing the SRA. Interestingly, attractive visits to the ascr #8 cue were only observed in 30-45% of the time, suggesting that the individual state of the animal plays a critical role in determining the behavioral response to the ascaroside, as seen in other ascaroside behavioral responses.

### Example 5: Peptidergic Signaling Drives Sex-Specific Ascr #8 Behavioral Response

Several neuropeptides of the FMRFamide-like-peptide (FLP) family have been implicated in the mechanosensory regulation of male-mating behavior. The genes encoding the neuropeptides flp-8, flp-10, flp-12, and flp-20 all suppress the number of turns around a hermaphrodite executed by a male prior to mating. Despite this enrichment of flp genes functioning in the mechanosensation of these male-specific behaviors, there has been no neuropeptide found to regulate the chemosensation of mating ascarosides. The example was designed to understand why an attractive concentration of a mating pheromone does not result in consistent attraction by investigating potential peptidergic signaling pathways that function in the sensation of ascr #8.

The initial screening of neuropeptides were focused on the FLP family. The him-8 lines of flp genes expressed in male-specific neurons were generated, specifically flp-3, flp-6, flp-12, and flp-19. To avoid confounding variables, the criteria for selection stipulated that outside of male-specific neurons, expression profiles was limited to a small number of neurons (flp-5 was therefore excluded as it exhibits expression in the pharyngeal muscle; while flp-21 and flp-22 are expressed in a large number of neurons outside of the male-specific expression profiles).

It was observed that loss of flp-3 strongly affected the ability of male C. elegans to respond to ascr #8, (FIG. 12A, FIG. 12B). The log(fold-change) of flp-3 is the only value significantly different than that seen in the wild-type (FIG. 12B). Interestingly, there was defect seen neither in flp-3 hermaphrodites, nor any other strain (FIG. 12D, FIG. 12E).

Because the defect in male response to ascr #8 was significant, and the SWAA was designed to detect attractive behaviors, it was sought to determine if flp-3 loss-of-function (lof) animals were in fact avoiding ascr #8. Using a previously described drop avoidance assay, forward moving animals were exposed to a drop of either vehicle control or ascr #8 and scored the avoidance index. Wild-type males did not avoid the cue, as expected for an attractive cue, while flp-3 lof males strongly avoided the pheromone (FIG. 12C). The hermaphroditic behavior was unaffected by the loss of flp-3. (FIG. 12F). Together, these results suggest that flp-3 functions to control the behavioral valence of the ascr #8 response to be attractive in a sex-specific manner; serving in males to suppress a basal avoidance behavior observed in hermaphrodites.

Rescue of flp-3 under a 4-kb region of its endogenous promoter was able to restore the behavioral valence of males to wild-type levels (FIG. 12G, FIG. 12I). While overexpression of neuropeptides can result in dominant negative phenotypes, expression of the flp-3 construct in wild-type animals did not alter wild-type behavioral response to ascr #8 (FIG. 12G, FIG. 12I).

To rule out an allele specific effect of the flp-3(pk361) mutation, which results in deletion of the entire coding sequence as well as 439 bp of upstream and 1493 bp of downstream genomic sequence flp-3(ok3265) was assayed, an in-frame deletion of the coding sequence that retains expression of two peptides produced by the flp-3 gene (FLP-3-1 and FLP-3-4). The flp-3(pk361) and flp-3(ok3265) mutant phenotypes were identical, confirming that the deletion in the pk361 allele did not cause any off-target effects, and that neither of the two peptides still encoded by the ok3625 allele (FLP-3-1 and FLP-3-4) are sufficient to rescue the mutant phenotype. Whether these peptides contribute to the ascr #8 response in another way remains to be elucidated.

### Example 6: FLP-3 Functions Specifically to Modulate the Ascr #8 Behavioral Response

While ascr #8 is a potent male-attracting pheromone, previous studies have shown that ascr #2, ascr #3, ascr #4 also function synergistically in attracting males. The CEM neurons that are required for ascr #8 sensation also function in ascr #3 sensation. While ascr #3 signal propagation is processed through the hub-and-spoke circuit centered around RMG, little is known about the mechanics of ascr #8 sensation outside of CEM involvement. To determine if flp-3 functions to regulate pheromone-mediated male attraction and avoidance in a general manner, or rather one specific to ascr #8, the response of wild-type and flp-3 lof males to ascr #3 was assayed, a cue for which behavioral valence has also recently been shown to be regulated in a sex-specific manner. It was observed that flp-3 lof males exhibited no defect in their attractive response to ascr #3, suggesting that its role is indeed specific to that of ascr #8 sensation.

Expression analysis of a FLP-3 translational fusion (pflp-3::flp-3::mCherry) confirmed previous expression analyses of the neuropeptide within male-specific spicule neurons (FIG. 12J-FIG. 12L). Transcriptional reporters have shown robust flp-3 expression in the amphid IL1 neurons, as well as the sensory PQR and the male-specific interneuron, CP9, although our translational fusion exhibited no PQR or CP9 expression (FIG. 12J-FIG. 12L). Previous studies employed 1-2 kb regions of promoter sequence driving GFP expression, while our construct employs a 4 kb region, thereby incorporating further regulatory elements that may restrict expression patterns. By including the full coding sequence in our translational fusion, we have also incorporated the regulatory elements found within the introns of the flp-3 gene.

Localization of mCherry within sensory cilia of male dorsal and ventral IL1 neurons was observed, as well as in puncta spanning their dendrites, consistent with peptide packing into dense core vesicles. Previous studies have observed dense core vesicles within, and being released from, dendritic arbors supporting the findings that FLP-3 is packaged within the dendrites of the IL1 neurons. An identical expression pattern within the IL1 cilia and dendritic puncta of hermaphrodites as well. Recent single-cell RNA-sequencing of the adult nervous system has again found more prolific expression of flp-3 throughout the nervous system, including most of the VC neurons. However, these studies were performed only in hermaphrodites, and were therefore unable to examine any male-specific changes in expression. In males, our flp-3 construct also exhibited male-specific tail expression in the SPD spicule neurons (FIG. 12J-FIG. 12L), and coupled with IL1 expression, completely rescued the attractive response to ascr #8 (FIG. 12G-FIG. 12I), suggesting a physiologically relevant site-of-release from this small subset of neurons.

Because the spicule neurons are exposed to the environment it was investigated whether they play a direct role in the sensation of ascr #8. To test this, ceh-30 lof males were assayed for their ability to avoid ascr #8. Male ceh-30 lof animals lack the male-specific CEM neurons responsible for ascr #8 sensation in the amphid region of the animal. The him-5 males did not avoid ascr #8. Males lacking CEM neurons also did not avoid ascr #8. However, with flp-3 still present in these animals, it may be that they are still able to sense the cue, but do not avoid it due to the presence of the neuropeptide. Therefore a ceh-30;flp-3 double mutant was generated and found that these animals still do not avoid the pheromone, confirming that the CEM neurons are the primary route of ascr #8 chemosensation which results in the male C. elegans behavioral response.

FLP-3 regulates attractive behavior to ascr #8 by activation of two evolutionarily divergent G protein-coupled receptors

The flp-3 gene encodes multiple peptides. Recent studies have uncovered a tenth peptide encoded by the gene; although this newest peptide does not contain the conserved GTMRFamide motif found in the remainder of flp-3 peptides (FIG. 13A). It was determined that the lysine-arginine sites flanking the individual peptides are processed specifically by the proprotein convertase encoded by the egl-3 gene, and not by aex-5 or bli-4, supporting previous studies that the egl-3 gene is the proprotein convertase involved in mating behaviors.

To better understand where the fully processed peptides act within the male-specific circuit, mutants were assayed for receptors that have previously shown activation upon FLP-3 peptide exposure. While activation of NPR-4 has been reported for only two peptides encoded by flp-3, NPR-5 and NPR-10 have been shown to respond to four and six flp-3 encoded peptides, respectively (FIG. 13A). Recent studies have considered NPR-4 and NPR-10 to be representative of one another due to their close phylogenetic relationship and separation by a recent gene duplication. However, in the testing of these mutants using SWAA, it was observed that npr-4 and npr-5 lof males respond similarly to him-8 males (FIG. 13B-FIG. 13D) while npr-10 lof animals exhibited a complete loss of attraction to the cue, as well as a partial avoidance phenotype matching that of flp-3 lof mutants (FIG. 13B-FIG. 13D).

Transgenic rescue by an NPR-10:: GFP translational fusion construct expressed under 1.6 kb of the endogenous promoter was able to restore wild-type levels off attraction in an npr-10 lof mutant background (FIG. 13C). This construct was also able to suppress the avoidance phenotype of npr-10 (FIG. 13D). Expression analysis of NPR-10::GFP revealed expression in both amphid and phasmid regions of the animals (FIG. 13D-FIG. 13F). Among these head neurons are the inner labial IL2 neurons, as well as their respective socket cells (ILso). NPR-10::GFP fluorescence was also observed in the ADL and ASG chemosensory neurons, cells which synapse onto AVD and AIA neurons, contributing to reversal control and turning circuitries, respectively. The localization of NPR-10 in the RMEL and RMEV neurons provides a direct input into neurons innervating muscle cells (FIG. 13E). Alongside expression in the interneuron AVK (FIG. 13E), which links the npr-10 circuitry to the backwards locomotion neuron AVE, AVF expression (FIG. 13E) links the circuit to the forward locomotion premotor interneuron, AVB.

NPR-10 expression was also observed in the B-class ray neurons in the male tail (FIG. 13F). Given the tight localization of these cells, it may be that NPR-10 is also present in the HOB neuron, which is impossible to decipher without further colocalization studies. However, the RnB neurons that express NPR-10 to sense SPD-secreted FLP-3 peptides heavily innervate the male-specific interneuron, EF1, which travels from the tail to synapse onto neurons in the head of the animal, including the forward locomotion neuron, AVB. Interestingly, the hermaphrodite tail exhibits expression in the dorso-rectal ganglion neurons DVA, DVB, DVC, and ALN, which is not observed in the male tail. These data suggest that NPR-10 plays sex-specific roles based on sex-specific expression patterns.

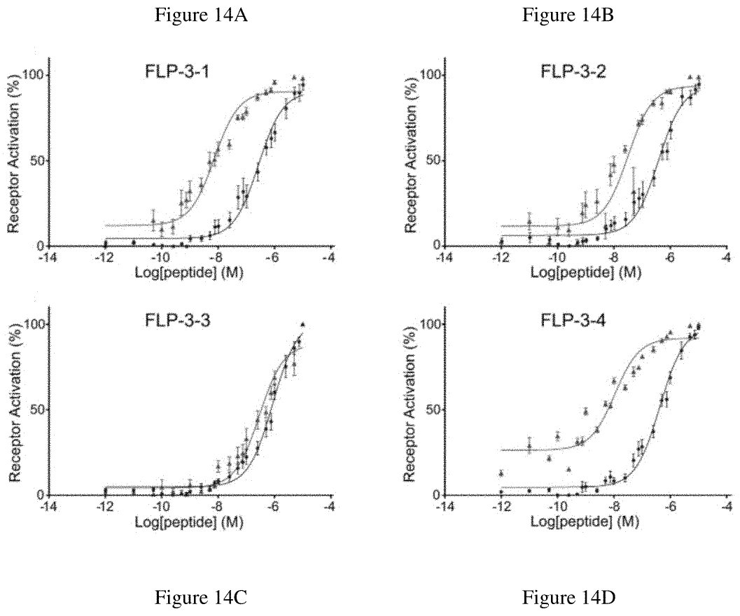

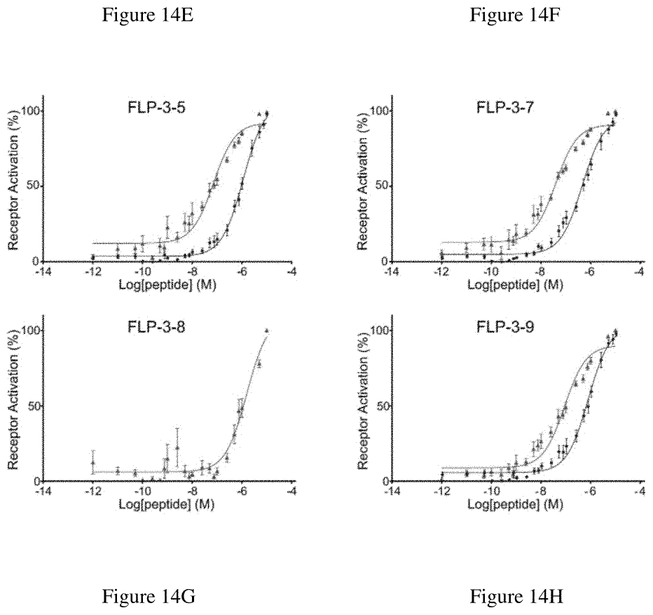

Using Chinese hamster ovarian (CHO) cell cultures stably expressing the promiscuous G protein, Gα16, and the calcium reporter, aequorin, it was observed that both isoforms of NPR-10 are activated by seven of the ten FLP-3 peptides, with half-maximal effective concentrations (EC₅₀) in the nM range (FIG. 14A-FIG. 14H, Table V). Peptide FLP-3-6 (EDGNAPFGTMKFamide) (SEQ ID NO: 8) did not activate NPR-10 in the assay. This peptide contains an R-to-K mutation within the C-terminal motif, which may explain the lack of receptor activation. Likewise, peptide FLP-3-10 (STVDSSEPVIRDQ) (SEQ ID NO: 9), which contains no sequence homology with any RFamide peptide (FIG. 14I) also failed to activate the receptor. Interestingly, FLP-3-8 (SADDSAPFGTMRFamide) did not activate either NPR-10A or NPR-10B, despite its conserved terminal amino acid sequence.

The lack of full avoidance phenotype observed in npr-10 lof mutants suggests that there are other FLP-3 receptors involved in regulating the ascr #8 avoidance behavior. Interestingly, while the human NPY/Drosophila NPF receptor NPR-10 is required for FLP-3 sensation, the Drosophila FR receptor homolog, FRPR-16, was also found to be reliably activated by FLP-3 peptides in vitro. This evolutionarily divergent receptor exhibited potencies in the 10-nanomolar range for seven of the peptides, and submicromolar for an eighth peptide (FIG. 14A-FIG. 14H). Again, FLP-3-6 and FLP-3-10 did not activate FRPR-16, supporting the notion that the terminal motif conserved in the remaining FLP-3 peptides is critical for receptor activation. Cells transfected with a control vector did not exhibit any activation following exposure to FLP-3 peptides, confirming that the activation observed is specific to receptor-ligand interactions with NPR-10 and FRPR-16.

A full-gene deletion of frpr-16 was generated using CRISPR mutagenesis (FIG. 15A-FIG. 15B. The frpr-16 lof males were assayed for their ability to respond to ascr #8. Males lacking frpr-16 exhibited a loss of attraction to ascr #8, as well as a partial avoidance phenotype, like that observed in npr-10 lof mutant animals (FIG. 15A-FIG. 15F). Interestingly, a double mutant containing both npr-10 and frpr-16 null alleles not only did not result in an additive effect in the avoidance phenotype, but in fact also suppressed the avoidance phenotype, suggesting that these receptors are nonredundant in their functions (FIG. 15E).

Rescue of FRPR-16 under 1.9 kb of its endogenous promoter was able to restore wild-type attractive behavior (FIG. 15C-FIG. 15D), as well suppress the avoidance phenotype (FIG. 15E). Localization of the mCherry fusion reporter was observed in the premotor interneurons responsible for reverse locomotory control: AVA, AVE, and AVD (FIG. 15F). The fluorescent protein was also seen anterior to the nerve ring in the gas-sensing BAG neuron (FIG. 15F). This expressing pattern was not sex specific, as a matching expression pattern was observed in hermaphrodites. Together, these data show that NPR-10 and FRPR-16 function as receptors for FLP-3 peptides.

### Example 7: Rescue of Individual FLP-3 Peptides by Feeding Reveals a Specific Subset of Active Peptides Required for Attractive Behavior

To identify which FLP-3 peptides are required for male avoidance of ascr #8, a peptide feeding approach https://www.nature.com/articles/s42003-021-02547-7-ref-CR59 was adopted, similar to RNAi feeding (FIG. 16A). Using Gateway Cloning technology, FLP-3 peptide coding sequences were inserted into a bacterial expression vector. As a control, first the FLP-3 peptides were tested that are still encoded in the ok3625 allele which are insufficient to drive the proper ascr #8 response in vivo: FLP-3-1 (SPLGTMRFamide) (SEQ ID NO: 10) and FLP-3-4 (NPLGTMRFamide) (SEQ ID NO: 11). Then flp-3 lof animals were reared on lawns of bacteria expressing the rescue constructs, and their progeny were assayed for avoidance (FIG. 16B-FIG. 16D). Neither FLP-3-1 nor FLP-3-4 were unable to rescue the avoidance phenotype on their own (FIG. 16B-FIG. 16D), supporting the flp-3(ok3625) data and further suggesting that these two peptides are insufficient to maintain wild-type behavior. This is surprising, as each peptide exhibits the highest affinities for FRPR-16, and two of the three highest affinities for NPR-10 (FIG. 14a-FIG. 14H).

The FLP-3-10 (STVDSSEPVIRDQ) were also tested, which exhibits a lack of consensus sequence and an inability to activate either NPR-10 or FRPR-16. The non-RFamide peptide was unable to rescue the avoidance phenotype (FIG. 16B). FLP-3-1 and FLP-3-4 differ in sequence only in their N-terminal amino acid (FIG. 13A, FIG. 14I). Similarly, a single amino acid change is all that distinguishes either peptide from FLP-3-2 (TPLGTMRFamide) (SEQ ID NO: 12), which exhibits the second highest affinity for NPR-10 (FIG. 14B). Therefore, FLP-3-2 was tested for its ability to rescue the flp-3 lof phenotype. Surprisingly, this peptide was able to abolish the avoidance phenotype observed in flp-3 lof animals (FIG. 16B), although it was not able to restore the animal's ability to be attracted to ascr #8 (FIG. 16C-FIG. 16D). The only difference being the presence of a threonine in that position of the peptide, which is here hypothesized that this may be the required component to suppress the avoidance behavior. Peptide FLP-3-9 (NPENDTPFGTMRFamide) (SEQ ID NO: 13) is a naturally occurring FLP-3 peptide that contains a threonine in the same location of the peptide. The N-terminus is capped with a NPEND (SEQ ID NO: 14) sequence, and the lysine conserved in FLP-3-1, 3-2, and 3-4 is mutated to a phenylalanine. However, when flp-3 lof animals were fed NPENDTPFGTMRFamide, (SEQ ID NO: 13) they not only displayed lack of avoidance to ascr #8, but also a full rescue of their ability to be attracted to the cue (FIG. 16A-FIG. 16H).

To further validate the ability of this threonine (T) to suppress avoidance of ascr #8, we tested the ability of FLP-3-7 (SAEPFGTMRFamide) (SEQ ID NO: 15) to restore wild-type behavior (FIG. 16E-FIG. 16H). It was observed that FLP-3-7 neither suppresses avoidance (FIG. 16H) nor drives attraction (FIG. 16F-FIG. 16G). However, when the ninth amino acid from the C-terminal, a glutamate (G), is replaced with a threonine, FLP-3-7T (FIG. 16E), it was observed to cause a suppression of the avoidance behavior (FIG. 16E), supporting the hypothesis that threonine is critical for suppressing male-specific avoidance of ascr #8.

While FLP-3-9, which contains this threonine, can drive attraction, it was observed that FLP-3-7T, which contains an SA sequence upstream of the threonine in place for FLP-3-9's NPEND sequence, is unable to drive attraction to ascr #8 (FIG. 16E-FIG. 16G). These results support the hypothesis that the NPEND sequence of FLP-3-9 is critical in regulating the attractive behavior.

Together, these data argue that the threonine in the ninth position from the C-terminus is critical for suppression of the basal avoidance response (FIG. 7A) as both FLP-3-2 and FLP-3-9, along with the modified FLP-3-7T peptide, were all capable of doing so, while FLP-3-1 and FLP-3-4 could not (FIG. 16A-FIG. 16H). Likewise, the NPEND sequence in FLP-3-9 may convey further specificity to the peptide, allowing it to drive attraction to the pheromone (FIG. 17A). It is not merely the presence of terminal amino acids that drives attraction, as the short sequence present in FLP-3-7T was unable to do so (FIG. 16A-FIG. 16H).

### Example 8: C. elegans Strains

The C. elegans strains N2 and CB4088 (him-5(e1490)) were obtained from the Caenorhabditis Genetics Center. The LSC1118 (trh-1(lst1118)) strain was generously provided by Isabel Beets at KU Leuven for the body morphology tests. The strains used in the Chemotaxis Assay were gifted by Prof. Shreekanth Chalasani, Salk Insitutute (IV302 (ins-6(tm2416); kyEx2595) at the Salk Institute, and Yun Zhang (ZC239 (ins-6(tm2416)II; yxEx175[Pins-6::ins-6; Punc-122::gfp]) at Harvard University. The UR954 (pdf-1(tm1886);him-5(e1490)) strain was provided by Douglas Portman at the University of Rochester.

Strains were obtained from the Caenorhabditis Genetics Center (University of Minnesota, Minn.), the National BioResource Project (Tokyo Women's Medical University, Tokyo, Jagan), Chris Li at City University of New York, Paul Sternberg at the California Institute of Technology, Ding Xue at University of Colorado Boulder, and Maureen Barr at Rutgers University. The novel allele of frpr-16 was generated via CRISPR editing using previously discussed methods. Strains were crossed with either him-5 or him-8 worms to generate stable males prior to testing.

### Example 9: Peptide Plasmid Design and Generation

DNA sequences encoding individual peptides were identified via a website wormbase. Sequences were flanked with the endogenous cleavage sites for the EGL-3 processing enzyme, which cleaves dibasic resides. Sequences encoding MRFGKR and KRK-STOP codons were therefore placed prior to, and following the peptide codon sequences, respectively. Finally, Gateway Cloning sites attB1 and attB2 sites were attached to the ends of the sequences. These final sequences (comprised of attB1::MRFGKR::peptide::KRK-STOP::attB2) were ordered from IDT (Integrated DNA Technologies) using their DNA Oligo and Ultramer DNA Oligo services, depending on the size of the oligo ordered. Both forward and reverse sequences were ordered (Table I).

Lyophilized oligos were prepared following IDT Annealing Oligonucleotides Protocol. The oligos were resuspended in Duplex Buffer (100 mM Potassium Acetate; 30 mM HEPES, pH 7.5; available from IDT), preheated to 94° C. to a final concentration of 40 μM. Complimentary oligo sequences were then mixed in equimolar ratios, and placed in a thermocycler at 94° C. for 2 minutes, prior to a stepwise cooling to room temperature.

Annealed oligos were used to perform a BP reaction with pDONR p1-2 donor vector to generate pENTRY clones (Gateway Cloning, ThermoFisher). Entry clones were then recombined with pDEST-527 (a gift from Dominic Esposito (Addgene plasmid #11518)) in LR reactions generating expression clones. The scramble control was generated in an identical manner, with the sequence between the cleavage sites being amplified from pL4440 (provided by Victor Ambros, University of Massachusetts Medical School, MA) (Table II). Purified plasmids were stored at −20° C. or −80° C. for long term storage.

TABLE 1
Sequences of forward and reverse oligos used in generation of plasmids containing
the peptide of interest for functional rescue of the neuropeptides

| Plasmid | Forward sequence | Reverse sequence |
|---|---|---|
| SCRAMBLE | GGGGACAAGTTTGTACAAAAAAGCAGGC | GGGGACCACTTTGTACAAGAAAGCTGG |
| | TGGATGCGCTTTGGAAAACGTAATTCGA | GTGTTATTTACGTTTCCTGCAGCCCGGG |
| | GCTCCACCGCGGTGGCGGCCGCTCTAGA | GGATCCACGCGCGTGGCTAGCCATGGAA |
| | ACTAGTGGATCCACCGGTTCCATGGCTA | CCGGTGGATCCACTAGTTCTAGAGCGGC |
| | GCCACGCGCGTGGATCCCCCGGGCTGCA | CGCCACCGCGGTGGAGCTCGAATTACGT |
| | GGAAACGTAAATAACACCCAGCTTTCTT | TTTCCAAAGCGCATCCAGCCTGCTTTTTT |
| | GTACAAAGTGGTCCCC (SEQ ID NO: 16) | GTACAAACTTGTCCCC (SEQ ID NO: 17) |
| TRH-1A | GGGGACAAGTTTGTACAAAAAAGCAGGC | GGGGACCACTTTGTACAAGAAAGCTGG |
| | TGGATGCGCTTTGGAAAACGTAGAGGAC | GTGTTATTTACGTTTTCCGAAAAGTTCTC |
| | GAGAACTTTTCGGAAAACGTAAATAACA | GTCCTCTACGTTTTCCAAAGCGCATCCA |
| | CCCAGCTTTCTTGTACAAAGTGGTCCCC | GCCTGCTTTTTTGTACAAACTTGTCCCC |
| | (SEQ ID NO: 18) | (SEQ ID NO: 19) |
| TRH-1B | GGGGACAAGTTTGTACAAAAAAGCAGGC | GGGGACCACTTTGTACAAGAAAGCTGG |
| | TGGATGCGCTTTGGAAAACGTCGTGCCA | GTGTTATTTACGTTTACCGAAAAGTTCA |
| | ATGAACTTTTCGGTAAACGTAAATAACA | TTGGCACGACGTTTTCCAAAGCGCATCC |
| | CCCAGCTTTCTTGTACAAAGTGGTCCCC | AGCCTGCTTTTTTGTACAAACTTGTCCCC |
| | (SEQ ID NO: 20) | (SEQ ID NO: 21) |
| INS-6 | CAATGCCACGAGCAAGTAGTGTTCCAG | CTGGTGCTGGAACACTACTTGCTCGTGG |
| | CACCAG (SEQ ID NO: 22) | CATTG (SEQ ID NO: 23) |
| PDF-1A | GGGGACAAGTTTGTACAAAAAAGCAGGC | GGGGACCACTTTGTACAAGAAAGCTGG |
| | TGGGTTCAGTTCGTAAAACGCAGCAACG | GTGTCAGCGTTTTCCGACAGCTGACAAT |
| | CCGAGCTTATCAACGGACTCATCGGAAT | TTTCCCAAATCCATTCCGATGAGTCCGTT |
| | GGATTTGGGAAAATTGTCAGCTGTCGGA | GATAAGCTCGGCGTTGCTGCGTTTTACG |
| | AAACGCTGACACCCAGCTTTCTTGTACA | AACTGAACCCAGCCTGCTTTTTTGTACA |
| | AAGTGGTCCCC (SEQ ID NO: 24) | AACTTGTCCCC (SEQ ID NO: 25) |
| PDF-1B | GGGGACAAGTTTGTACAAAAAAGCAGGC | GGGGACCACTTTGTACAAGAAAGCTGG |
| | TGGGTTCAGTTCGTAAAACGCTCAAACG | GTGTCATCGTCGACCAGCTCCAGACAAT |
| | CGGAACTTATCAACGGTCTTCTCAGCAT | TTGTTGAGGTTCATGCTGAGAAGACCGT |
| | GAACCTCAACAAATTGTCTGGAGCTGGT | TGATAAGTTCCGCGTTTGAGCGTTTTAC |
| | CGACGATGACACCCAGCTTTCTTGTACA | GAACTGAACCCAGCCTGCTTTTTTGTAC |
| | AAGTGGTCCCC (SEQ ID NO: 26) | AAACTTGTCCCC (SEQ ID NO: 27) |

TABLE II
Processed peptide amino acid sequences used for bacterial rescue
experiments.

| Peptide | Sequence (COOH-x-NH2) |
|---|---|
| SCRAMBLE | NSKLHRGGGRSRTSGSTGSMASHARGSPGLQ (SEQ ID NO: 28) |
| TRH-1A | GRELF (SEQ ID NO: 29) |
| TRH-1B | ANELF (SEQ ID NO: 30) |
| INS-6 | VPAPGETRACGRKLISLVMAVCGDLCNPQEGKDIATECCGNQCSDDYIRS |
| | ACCP (SEQ ID NO: 31) |
| PDF-1A | VQFVKRSNAELINGLIGMDLGKLSAVGKR (SEQ ID NO: 32) |
| PDF-1B | VQFVKRSNAELINGLLSMNLNKLSGAGRR (SEQ ID NO: 33) |

### Example 10: Fusion Constructs

DNA for the flp-3, npr-f, and frpr-6 promoter and coding regions were isolated from C. elegans genomic DNA via PCR.

In generating the flp-3 rescue product, PstI and BamHI restriction sites added onto the isolated fragments were introduced through primer design. PCR amplicons and the Fire GFP Vector, pPD95.75 (kindly provided by Josh Hawk, Yale University, CT), were digested with PstI and BamHI enzymes. Products were ligated together to generate JSR #DKR18 (pflp-3::flp-3::GFP). The flp-3 expression analysis construct, JSR #DKR34 (pflp-3::flp-3::SL2::mCherry) was generated by Genewiz. The promoter-gene fragment of npr-10 was generated by Gibson Assembly to GFP (from pPD95.75) and as a linear fusion. The rescue-fusion construct of frpr-6 was achieved by fusing the promoter and gene sequence to mCherry isolated from JSR #DKR34 via Gibson Assembly. The primer and ultramer sequences are listed in Table III.

TABLE III
List of primer and ultramer sequences

| Primer/ | | |
|---|---|---|
| Ultramer Name | Primer/Ultramer Sequence | SEQ ID NO |
| pflp-3 Forward | GACTCTGCAGcatttccaagacacatttgacg | SEQ ID NO: 34 |
| pflp-3 Forward | GACTGGATCCttttccaaagcgcatggtt | SEQ ID NO: 35 |
| pnpr-10 Forward | gtttgttttccggcactttc | SEQ ID NO: 36 |
| npr-10 | AGTCGACCTGCAGGCATGCAAGCTaatt | SEQ ID NO: 37 |
| Reverse_overhang | gaacttggaatcgtggtagt | |
| pnpr-10 | cggcactttcctcatttttc | SEQ ID NO: 38 |
| Forward#2 | | |
| pfrpr-16 Forward | accgatttctgatcgacgtg | SEQ ID NO: 39 |
| frpr-16 | CAGCAGTTTCCCTGAATTAAAATTAaac | SEQ ID NO: 40 |
| Reverse_overhang | aattgccgg agcttttc | |
| pfrpr-16 | ttctgatcgacgtgttggtt | SEQ ID NO: 41 |
| Forward#2 | | |
| GFP_C | AGCTTGCATGCCTGCAGGTCGACT | SEQ ID NO: 42 |
| GFP_D | AAGGGCCCGTACGGCCGACTAGTAG | SEQ ID NO: 43 |
| GFP/dsRed_D#2 | GGAAACAGTTATGTTTGGTATATTGGG | SEQ ID NO: 44 |
| dsRed_E | TAATTTTAATTCAGGGAAACTGCTG | SEQ ID NO: 45 |
| dsRed_F | AAAGTTGgaaacagttatgtttgg | SEQ ID NO: 46 |
| SCRAMBLE | GGGGACAAGTTTGTACAAAAAAGCAG | SEQ ID NO: 47 |
| Forward | GCTGGATGCGCTTTGGAAAACGTaattcg | |
| | aagctccaccgcggtggcggccgctctagaactagtggatcc | |
| | accggttccatggctagccacgcgcgtggatcccccgggct | |
| | gcaggAAACGTaaataaCACCCAGCTTTCT | |
| | TGTACAAAGTGGTCCCC | |
| FLP-3-1 Forward | GGGGACAAGTTTGTACAAAAAAGCAG | SEQ ID NO: 48 |
| | GCTGGATGCGCTTTGGAAAACGTtctcca | |
| | ctgggaacaatgcgctttggcAAACGTaaataaCAC | |
| | CCAGCTTTCTTGTACAAAGTGGTCCCC | |
| FLP-3-2 Forward | GGGGACAAGTTTGTACAAAAAAGCAG | SEQ ID NO: 49 |
| | GCTGGATGCGCTTTGGAAAACGTactcca | |
| | ttgggaactatgcgttttggaAAACGTaaataaCACC | |
| | CAGCTTTCTTGTACAAAGTGGTCCCC | |
| FLP-3-4 Forward | GGGGACAAGTTTGTACAAAAAAGCAG | SEQ ID NO: 50 |
| | GCTGGATGCGCTTTGGAAAACGTaaccct | |
| | cttggaaccatgcgctttggaAAACGTaaataaCACC | |
| | CAGCTTTCTTGTACAAAGTGGTCCCC | |
| FLP-3-9 Forward | GGGGACAAGTTTGTACAAAAAA | SEQ ID NO: 51 |
| | GCAGGCTGGATGCGCTTTGGAAAACGTaatcct | |
| | gagaacgacacaccattcggaacaatgagatttggaAAA | |
| | CGTaaataaCACCCAGCTTTCTTGTACAA | |
| | AGTGGTCCCC | |
| FLP-3-10 | GGGGACAAGTTTGTACAAAAAA | SEQ ID NO: 52 |
| Forward | GCAGGCTGGATGCGCTTTGGAAAACG | |
| | TtctactgttgattcttcggagcccgtcattcgtgatcagAA | |
| | ACGTaaataaCACCCAGCTTTCTTGTACA | |
| | AAGTGGTCCCC | |
| FLP-3-7 Forward | GGGGACAAGTTTGTACAAAAAA | SEQ ID NO: 53 |
| | GCAGGCTGGATGCGCTTTGGAA | |
| | AACGTagtgcagagccattcggtactatgcgttt | |
| | tggaAAACGTAAATAACACCCAG | |
| | CTTTCTTGTACAAAGTGGTCCCC | |
| FLP-3-7T Forward | GGGGACAAGTTTGTACAAAAAA | SEQ ID NO: 54 |
| | GCAGGCTGGATGCGCTTTGGAA | |
| | AACGTagtgcaactccattcggtactatgcgtttt | |
| | ggaAAACGTAAATAACACCCAGC | |
| | TTTCTTGTACAAAGTGGTCCCC | |
| frpr-16-1 SeqValR | ggtatctgtggtttatgtcggatag | SEQ ID NO: 55 |
| frpr-16-2 SeqValR | gaacggcatacgttcaggata | SEQ ID NO: 56 |
| frpr-16-2 WT F | aaactaccctgtgcgattagg | SEQ ID NO: 57 |
| frpr-16-1 WT R | CTTCCATACGTCAAGCACTTTC | SEQ ID NO: 58 |
| pMYO-2 SEC | ccctcaatgtctctacttgt | SEQ ID NO: 59 |
| Insertion | | |
| NeoR-SEC | TTCCTCGTGCTTTACGGTATCG | SEQ ID NO: 60 |
| Insertion | | |
| frpr-16-1 SeqVal F | actttcaggcgaacacatact | SEQ ID NO: 61 |

### Example 11: Peptide Plate Preparation

The expression clone for both the peptide of interest and the control was transformed into competent DH5α cells (NEB® [New England Biolabs] 5-alpha Competent E. coli (High Efficiency) Cat #C29871).

Peptide-expressing cultures on LB agar plates (10 g/L NaCl, 10 g/L Tryptone, 5 g/L Yeast Extract, 5 g/L Agar) containing 100 μg/μL ampicillin (AMP), were stored at 4° C. for up to 2 months. From this plate, single colonies were selected for growth in 5 mL of LB media containing ampicillin at 37° C. for 16 hours.

The optical density of the grown cultures (OD600) was adjusted to 1.0. 75 μL of 1.0 OD600 peptide-expressing culture was plated onto 60 mm NGM agar plates (50 mM NaCl. 25 mM KPO4 (pH 6.0), 1 mM MgSO4, 1 mM CaCl2, 5 μg/μL cholesterol, 17 g/L agar, 2.5 g/L peptone) containing 100 μg/μL AMP and 1 mM isopropyl-β-D-thiogalacto side (IPTG) at room temperature for at least 8 hours prior to use, for no longer than 1 week.

### Example 12: Peptide Feeding

All animals were maintained on bacterial lawns of OP50 E. coli, at 20° C., on NGM agar plates until the start of experiments. Mutant or control animals were transferred onto lawns of DH5α E. coli expressing either scramble peptide or the rescue peptide-of-interest on NGM plates containing 100 μg/μL AMP and 1 mM IPTG. Animals were reared on the peptide-expressing lawns for at least 48 hours at 20° C. before assaying for rescue of mutant phenotype at the appropriate developmental stage.

### Example 13: Transgenic Animals

CB1489 animals were injected with JSR #DKR18 (pflp-3::flp-3::GFP at 20 ng/μL), using punc-122::RFP (at 20 ng/μL) (kindly provided by Sreekanth Chalasani at the Salk Institute, CA) as a co-injection marker to generate JSR81 (him-8(e1489);worEx17[pflp-3::flp-3::GFP; punc-122::RFP]). JSR81 was then crossed with JSR99 to generate JSR109 (flp-3(pk361);him-8(e1489);worEx17[pflp-3::flp-3::GFP; punc-122::RFP]).

PS2218 animals were injected with JSR #DKR34 (pflp-3::flp-3::SL2::mCherry at 25 ng/μL), using punc-122::GFP (at 50 ng/μL) as a co-injection marker to generate JSR119 (dpy-20(e1362);him-5(e1490);syls33[HS.C3 (50 ng/μL)+pMH86 (11 ng/μL)];worEx21[pflp-3::flp-3::SL2::mCherry; punc-122::GFP]).

JSR102 animals were injected with a linear fusion product (pnpr-10::npr-10::GFP at 25 ng/μL), alongside punc-122::RFP (at 50 ng/μL) as a co-injection marker to generate JSR126 (npr-10(tm8982);him-8(e1489);worEx37[pnpr-10::npr-10::GFP, punc-122::RFP]). This was crossed with PT2727 (myIS20 [pklp-6::tdTomato+pBX], a gift from Maureen Barr) to generate JSR138 (myIS20 [pklp-6::tdTomato, pBX]; npr-10(tm8982);him-8(e1489);worEx37[pnpr-10::npr-10::GFP, punc-122::RFP]).

JSR103 animals were injected with a linear fusion product (pfrpr-16::frpr-16::SL2::mCherry at 25 ng/μL), alongside punc-122::GFP (at 50 ng/μL) as a co-injection marker to generate JSR133 (frpr-16(gk5305[loxP+pmyo-2::GFP::unc-54 3′ UTR+prps-27::neoR::unc-54 3′ UTR+loxP]);him-8(e1489);worEx41[pfrpr-16::frpr-16::SL2::mCherry, punc-122::GFP]).

Injections for JSR81 were generously performed by the Alkema Lab at UMass Medical School. Injections for JSR119, JSR126, and JSR133 were performed by In Vivo Biosystems (formerly NemaMetrix).

### Example 14: Chemical Compounds

The ascarosides ascr #3 and ascr #8 were synthesized as described previously in Pungaliya, C. et al. Proc. Natl Acad. Sci. USA 106, 7708-7713 (2009) and Srinivasan, J. et al. Nature 454, 1115-1118 (2008). Peptides used in in vitro GPCR activation assays were synthesized by GL Biochem Ltd.

### Example 15: Size Comparison (trh-1)

The body morphology different of trh-1 mutants was compared to wild-type worms by body volume measurements. Wild-type (N2) and trh-1 animals were synchronized by embryonic starvation following alkaline hypochlorite protocols. Ten gravid hermaphrodites from OP50 E. coli plates were picked into 30 μL drops of alkaline hypochlorite solution (20% sodium hypochlorite, 500 mM KOH). Two drops were placed on either side of an unseeded NGM plate, resulting in 20 animals per plate. Plates were then incubated for 24 hours at 20° C. before L1 larval animals washed with 1 mL M9 (22 mM KH₂PO₄, 42.25 mM Na₂HPO₄, 85.5 mM NaCl, 1 mM MgSO₄) per unseeded NGM plate. Allow L1 worms to settle into pellet before removing M9 supernatant. Repeat washing step 2 more times. Plate worm pellet onto NGM with 100 μg/μL AMP and 1 mM IPTG plates containing 75 μL peptide lawns.

After at 48 hours at 20° C., animals were recorded using a Basler acA2500-14 uM camera using WormLab software (WormLab 4.1; MBF Bioscience, 2017) for 65 seconds. Worm length, width, and area were calculated within the WormLab software (FIG. 5C-FIG. 5H). As the WormLab worm width metric is an average of cross-sections throughout the length of each worm, this was used to calculate the volume of a worm as a cylinder, with the Volume=π*(0.5*width)²*length (FIG. 2A-FIG. 2B, FIG. 5A-FIG. 5B).

### Example 16: Chemotaxis Assay (ins-6)

A salt chemotaxis assay was performed as previously described in Leinwand, S. G. & Chalasani, S. H. Nature neuroscience 16, 1461-1467 (2013); Bargmann, C. I. & Horvitz, H. R. Neuron 7, 729-742 (1991) to test the functionality of ins-6 after rescue-by-feeding. Wild-type (N2) and ins-6 worms were fed either scramble or INS-6 peptide passing 4-6 L4 larval onto an NGM plate containing 100 μg/μL AMP and 1 mM IPTG with 75 μL of peptide OD₆₀₀=1.0. These worms were grown at 20° C. for about 4 days until the progeny of the initial L4 worms were young adults.

The day prior to testing, a thin, 10 mL layer of agar was added to 10 cm petri dishes to generate a 10 cm Chemotaxis Plates (5 mM KPO₄ (pH 6.0), 1 μM MgSO₄, 1 μM CaCl₂), 20 g/L agar, and 8 g/L Difco Nutrient Broth). To prepare the “high salt” plates, 750 mM NaCl was added prior to plate pouring. The high salt plate were stored at 4° C. for up to one month, while the Chemotaxis Plates were made one day prior to testing. To create the high and low salt plugs, the back end of a Pasteur pipette was used to punch a 5 mm plug out of each plate. These 2 plugs were placed on opposite edges of the plate. A 10 mm radius was be drawn around the location of the plugs (FIG. 1). Plates were stored, lightly covered, overnight at room temperature (˜20° C., <40% humidity).

The day of the assay, young adult worms, either control worms fed OP50 E. coli or peptide-fed worms, were washed off peptide plates with M9. Worms were allowed to settle by gravity for 4 minutes before removing the supernatant, washing the worms. Worms were then washed with Chemotaxis Buffer (5 mM KPO₄ (pH 6.0), 1 mM MgSO₄, 1 mM CaCl₂)) three times.

To prepare the Chemotaxis plates for the assay, high and low salt plugs were removed with forceps, and the plates spotted with 1 μL of 0.5 M sodium azide (NaN₃). Approximately 30 μL of worms in Chemotaxis Buffer were spotted onto the edge of the agar, between the location of the high and low salt gradient (FIG. 6A-FIG. 6B). Worms were left to chemotax for 1 hour before scoring the number of worms within the 10 mm radii of high and low salt areas.

### Example 17: Excursion Assay (pdf-1)

The mate searching behavior of pdf-1 mutants was quantified in a food leaving assay. him-5 animals were used as wild-type control to ensure the presence of males. him-5 and pdf-1 animals were fed OP50, scramble peptide, PDF-1A, PDF-1B, or a 1:1 mixture of PDF-1A and PDF-1B cultures. Four to six larval L4 hermaphrodites were passed onto an NGM plate containing 100 μg/μL AMP and 1 mM IPTG with 75 μL of peptide. These worms were grown at 20° C. for about 4 days until the male progeny of the initial L4 worms were young adults. The day prior to the assay, young adult male pdf-1 or him-5 worms were singled onto 60 mm plates containing lawns of either OP50 (NGM plates) or appropriate peptide cultures (NGM containing 100 μg/μL AMP and 1 mM IPTG). To determine the mate searching effects of pdf-1, we performed a food-leaving assay as described previously in Barrios, A., et al., Nat Neurosci 15, 1675-1682 (2012); Lipton, J., et al., The Journal of neuroscience: the official journal of the Society for Neuroscience 24, 7427-7434 (2004); Ryan, D. A. et al. Curr Biol 24, 2509-2517 (2014), with modifications. In this assay, individuals were placed on a small food spot, and track patterns were scored at the indicated times. Assay plates were prepared one day prior to assaying on 100 mm plates with 10 mL of Leaving Assay media (25 mM KPO₄ (pH 6.0), 1 mM MgSO₄, 1 mM CaCl₂), 50 mM NaCl, 2.5 g/L Peptone, 17 g/L Agar). On center of plate, 7 μL of OP50 was added. Plates were lightly covered and stored at room temperature overnight (˜20° C., <40% humidity).

On the day of the assay, a single male was plated on the assay plate and placed in a dark, 20° C. incubator. At 2, 6, and 24 hours after the worms are plated, plates were scored for male worm tracks. Scored were separated into binned ranges. “Never left food” indicates the absence of tracks outside the food spot. “Minor excursion” indicates that tracks were observed not beyond 10 mm from the food. “Major excursion” indicates the presence of tracks past the 1 cm boundary. The food leaving behavior was quantified at three different time points (2, 6, and 24 hours). (FIG. 4A, FIG. 7A, Table IV).

The mate searching defect of pdf-1 was quantified as a Probability of Leaving per hour (P_{L}) (FIG. 4B, FIG. 7B). In this method, the analysis of the leaving behavior was reduce to a single value, by scoring the worms leaving the food lawn as Leavers (traveling beyond 35 mm from the food source) or Non-Leavers (did not travel further than 35 mm from center of food).

TABLE IV
Probability of Leaving PL for each peptide feeding condition
in him-5 and pdf-1 males.

| Genotype | Treatment | P_{L} (confidence interval) | [n] |
|---|---|---|---|
| him-5 | OP50 | 0.06553819 (0.05587592, 0.07687131) | [72] |
| him-5 | SCRAMBLE | 0.07727273 (0.06531852, 0.09141473) | [55] |
| him-5 | PDF-1A | 0.06979167 (0.05892111, 0.08268776) | [60] |
| him-5 | PDF-1B | 0.08712121 (0.0754002, 0.1006643) | [66] |
| him-5 | PDF-1A + B | 0.07720588 (0.06483647, 0.09193512) | [51] |
| pdf-1 | OP50 | 0.01785714 (0.0134571, 0.02369585) | [84] |
| pdf-1 | SCRAMBLE | 0.03537736 (0.02746855, 0.04556329) | [53] |
| pdf-1 | PDF-1A | 0.03286638 (0.02657212, 0.04224128) | [58] |
| pdf-1 | PDF-1B | 0.05821078 (0.04760713, 0.07117621) | [61] |
| pdf-1 | PDF-1A + B | 0.04473039 (0.03556126, 0.0562637) | [51] |

### Example 18: Size Comparisons

Two to four plates were recorded per day for at least three days to generate data sets. Control plates of N2 and trh-1 animals raised on the scramble peptide were grown alongside every rescue feeding. Each “worm track” was considered an n=1. Tracks were excluded if (1) worm tracks less than 10 seconds, or (2) worms were further than two standard deviations from the mean metric. Following tests for normality, comparisons were made using either Mann-Whitney tests for direct comparisons between N2 and trh-1 animals reared on OP50, or Kruskal-Wallis non-parametric ANOVAs and Dunn's multiple comparisons tests for animals raised on peptide feeding strains. Comparisons were made for metrics: length (FIG. 5C-FIG. 5D), width (FIG. 5e-FIG. 5F), area (FIG. 5G-FIG. 5H), and volume (FIG. 2A-FIG. 2B, FIG. 5A-FIG. 5B).

### Example 19: Chemotaxis Assay

Young adult hermaphrodites are tested in the Chemotaxis Assay with no more than three replicates per day, over a minimum of three days, with 100-200 worms/assay plate. The 19 Chemotaxis Index (CI) was calculated as follows:

$$CI = \frac{\# worms in high salt - \# of worms in low salt}{total \# worms}$$

At least 10 plates were assayed in each condition: OP50 control, scramble fed, and INS-6 fed. Plates were excluded from final calculations if (1) the total number of worms on the assay plate was <20, or (2) if the plate CI was greater than two standard deviations from the average CI. Conditions were compared using Two-tailed t-tests with samples of equal variable to compare between two conditions with small samples or ANOVA followed by Bonferroni's multiple comparison for multiple condition comparisons.

### Example 20: Excursion Assay

The assay was performed across at least two days per condition, with at least n=50, wherein each assay plate with one male is equal to n=1. In all experiments, the investigators were blinded to genotype. Worm in the >35 mm from the center of food bin were scored as “leavers”. The probability of leaving per hour (Probability of Leaving (PL)) was calculated using an R script developed in Barrios et al., with assistance by (Personal Communications to Barrios, 2020). PL was calculated by the “hazard obtained by fitting an exponential parametric survival model to the censored data using maximum likelihood” (Table IV). The PLh-1 values for all pdf-1 animals fed scramble or peptide rescue were compared to him-5 worms fed scramble using an ANOVA followed by Bonferroni's multiple comparison (FIG. 4B, FIG. 7C).

### Example 21: Single Worm Assay

The outer forty wells of a 48-well suspension culture plate (Olympus Plastics, Cat #: 25-103) were seeded with 200 μL of standard NGM agar. To prepare the plates for the assay, they were acclimated to room temperature, at which point each well was seeded with 65 μL of OP50 E. coli. The assay plates were then transferred to a 37° C. incubator with the lid tilted for 4 h to allow the bacterial culture to dry on the agar. Once the bacterial culture dried, the lid was replaced the plate was stored at 20° C. until used in the assay. Fifty to sixty L4 worms were segregated by sex and stored at 20° C. for 5 h to overnight to be assayed as young adults. 0.8 μL of either vehicle control or ascaroside #8 was placed in the center of the well corresponding to that condition within the quadrant being tested, following a random block design (FIG. 11A). A single worm was placed in each of the 10 wells to be assayed, and the plate was transferred to a light source and camera and recorded for 15 m. This process was repeated for all four quadrants. Each strain and sex were assayed over five plates assayed on at least three different days.

### Example 22: Raw Dwell Time

Raw dwell time values were calculated by subtracting the time a worm exited the cue (center of the well in spatial controls), from the time it entered, as in the SRA. This was determined per visit, and the average dwell time was calculated for each animal in the quadrant. Averages of the four-quadrant means were determined per plate, and a minimum of five plates were assayed per strain/condition. The mean raw dwell time across five plates was calculated and used for statistical analyses and graphical display.

### Example 23: Log(Fold-Change)

The average dwell time in the ascaroside was divided by the average dwell time within the vehicle control per plate to generate a fold-change. To transform the data, the log of this fold-change was taken, and the average log(fold-change) was used for statistical analyses and graphical display.

### Example 24: Visit Count

The number of visits per-worm was calculated, and the average visit count determined per quadrant, and per plate. The average visit count across five plates was calculated and used for statistical analyses and graphical display.

### Example 25: Percent Attractive Visits

An “attractive visit” was first determined for each plate as any visit greater than two standard deviations above the mean dwell time within the vehicle control for that plate. Any individual visit meeting this threshold was scored as a “1”, and any below was scored a “0”. The percent visits per-worm that were attractive was determined, and the average of each quadrant taken. The four-quadrant values were then averaged to generate plate averages. The average percent of attractive visits across five plates was calculated and used for statistical analyses and graphical display.

### Example 26: Avoidance Assay

Assays were performed as described previously in Chute, C. D. & Srinivasan, J. Semin. Cell Dev. Biol. 33, 18-24 (2014); Chute, C. D. et al. Nat. Commun. 10, 3186 (2019); and Hilliard, M. A. et al., EMBO J. 23, 1101-1111 (2004). Fifty to sixty L4 worms were segregated by sex and stored at 20° C. for 5 h to overnight to be assayed as young adults. One to four hours prior to the assay, the lids of unseeded plates were tilted to allow any excess moisture to evaporate off the plates. At the time of the assay, 10 or more animals were transferred onto each of the dried, unseeded plates. A drop of either water or 1 μM ascr #8 was placed on the tail of forward moving animals, and their response was scored as either an avoidance response, or no response. The total number of avoidances was divided by the total number of drops to generate an avoidance index for that plate. This was repeated for at least 10 plates over at least three different days.

### Example 27: Spot Retention Assay Statistical Analysis

Statistical comparisons within each strain were made by Paired t-tests. For comparisons between strains/conditions, the data was transformed as described previously in Zhang, Y. K et al., J. Organic Biomol. Chem. (2019). The data were transformed to have only non-zero data for the calculation of fold-changes. This was done using a Base 2 Exponentiation (2ⁿ, where n is equal to the dwell time). The log (base 2) of the fold-changes of these transformed values was used to allow for direct comparisons between strains of the same background (i.e., him-5 and osm-3;him-5) using a Student's t-test. p values are defined in respective figure captions, with thresholds set as: *p<0.05, **p<0.01, ***p<0.001, ****p<0.0001.

### Example 28: Single Worm Assay Statistical Analysis

Statistical comparisons within each strain/sex (spatial, vehicle, ascaroside) were made by Repeated Measured ANOVA with the significance level set at 0.05, followed by multiple comparisons using Bonferroni correction. For comparisons between strains/sexes, the spatial control dwell times were compared using a one-way ANOVA followed by a Dunnett's correction to confirm that mutations of interest had no effect on the amount of time animals naturally spent in the center of the well. To directly compare strains, a fold-change was calculated by dividing the ascaroside by vehicle dwell times for each assay. This was then transformed by taking the log (base 10) of the fold-change. Comparisons were then made by one-way ANOVA followed by multiple comparisons using Dunnett's correction. p values are defined in respective figure captions, with thresholds set as: *p<0.05, **p<0.01, ***p<0.001, ****p<0.0001.

### Example 29: Avoidance Assay

Statistical comparisons within each strain were made by paired t-test against a significance level set at 0.05. For comparisons between strains/conditions, comparisons were made by One-Way ANOVA, followed by multiple comparisons using Bonferroni correction. p values are defined in respective figure captions, with thresholds set as: *p<0.05, **p<0.01, ***p<0.001, ****p<0.0001.

### Example 30: In Vitro GPCR Activation Assay

The GPCR activation assay was performed as previously described Van Sinay, E. et al. Proc. Natl Acad. Sci. USA 114, E4065-e4074 (2017) and Peymen, K. et al. PLoS Genet. 15, e1007945 (2019). Briefly, npr-10 and frpr-16 cDNAs were cloned into the pcDNA3.1 TOPO expression vector (Thermo Fisher Scientific). A CHO-K1 cell line (PerkinElmer, ES-000-A24) stably expressing apo-aequorin targeted to the mitochondria (mtAEQ) and human Gα16 was transiently transfected with the receptor cDNA construct or the empty pcDNA3.1 vector using Lipofectamine LTX and Plus reagent (Thermo Fisher Scientific). Cells were shifted to 28° C. one day later and allowed to incubate for 24 h. On the day of the assay, cells were collected in BSA medium (DMEM/Ham's F12 with 15 mM HEPES, without phenol red, 0.1% BSA) and loaded with 5 mM coelenterazine h (Thermo Fisher Scientific) for 4 h at room temperature. The incubated cells were then added to synthetic peptides dissolved in DMEM/BSA, and luminescence was measured for 30 s at 496 nm using a Mithras LB940 (Berthold Technologies) or MicroBeta LumiJet luminometer (PerkinElmer). After 30 s of readout, 0.1% triton X-100 was added to lyse the cells, resulting in a maximal calcium response that was measured for 10 s. After initial screening, concentration-response curves were constructed for HPLC-purified FLP-3 peptides by subjecting the transfected cells to each peptide in a concentration range from 1 pM to 10 μM. Cells transfected with an empty vector were used as a negative control. Assays were performed in triplicate on at least two independent days. Concentration-response curves were fitted using Prism v. 7 (nonlinear regression analysis with a sigmoidal concentration-response equation).

### Example 31: Generation of a Null Frpr-16 Mutant by CRISPR Mutagenesis

The frpr-16 CRISPR/Cas9 knockout was provided by the Vancouver node of the International C. elegans Consortium. The mutation was generated following previously described techniques as in Au, V. et al. G3 (Bethesda) 9, 135-144 (2019). A 1685 bp region containing the coding sequence, as well 52 bp upstream and 60 bp downstream, was removed from the genome, and replaced with a trackable cassette containing pmyo-2::GFP and a neomycin resistance gene (FIG. 15A and FIG. 15B). The flanking sequences of the mutated sequence are TCATAATTGTTTGTTTGACAAAAACCGGGA (SEQ ID NO: 62) and GGTGGAAACGGAAATGAAAGAAAAAACCGA (SEQ ID NO: 63). PCR confirmation of gene replacement with cassette was performed via four sets of PCR reactions checking the upstream insertion site and the downstream insertion site in the mutant strain, and a test for wild-type sequence in both mutant and wild-type strains. A band is present on the gel in wild-type samples, with no band present in the mutant, as a primer sequence is removed with the cassette insertion. See Table III for primer sequences.

### Example 32: Peptide Rescue

SCRAMBLE control or FLP-3 peptide constructs were grown overnight in LB media containing 50 μg/μL ampicillin at 37° C. and diluted to an OD₆₀₀ of 1.0 prior to seeding on NGM plates containing 50 μg/μL ampicillin and 1 mM IPTG. The 75 μL lawn was left to dry and grow overnight at room temperature before three L4 animals were placed on the plates. Males were selected for testing in the same manner as described above but were isolated onto plates also seeded with the same peptide on which they had been reared. Animals were then assayed using either the avoidance assay or single worm assay.

### Example 33: Imaging

Animals were mounted on a 2% agar pad and paralyzed using 1 M sodium azide on a microscope slide, as described previously in Chute, C. D. et al. Nat. Commun. 10, 3186 (2019). Images for amphid flp-3 expression were acquired using a Zeiss LSM700 confocal microscope. Final images were obtained using a ×63 oil objective with a ×1.4 digital zoom, for a final magnification of ˜×90. Tail images were acquired using a Zeiss Apotome using a ×40 oil objective. Images of npr-10 and frpr-16 expression were acquired using a Zeiss LSM510 Meta inverted confocal microscope. Final images of npr-10 were obtained using a ×63 oil objective with a ×0.8 digital zoom, for a final magnification of ˜×50. Final images of frpr-16 were obtained at either ×20 (air objective) or ×63 (oil) with a 2× digital zoom, for a final magnification of ˜×125.

TABLE V
EC₅₀ values and 95% Confidence Intervals for FLP-3 peptide
activating NPR-10B and FRPR-16

| | | NPR-10 | | FRPR-16 | |
|---|---|---|---|---|---|
| | FLP-3 Peptide | EC₅₀ | 95% CI | EC₅₀ | 95% CI |
| 3-1 | SPLGTMRFamide | 262.1 nM | 189.7 nM to 359.8 nM | 7.4 nM | 5.2 nM to 10.6 nM |
| 3-2 | TPLGTMRFamide | 366.5 nM | 270.4 nM to 491.3 nM | 31.7 nM | 19.0 nM to 51.2 nM |
| 3-3 | EAEEPLGTMRFamide | 866.7 nM | 706.6 nM to 1.1 μM | 288.3 nM | 203.7 nM to 402.3 nM |
| 3-4 | NPLGTMRFamide | 427.1 nM | 351.7 nM to 516.3 nM | 10.7 nM | 7.6 nM to 15.3 nM |
| 3-5 | ASEDALFGTMRFamide | 1.1 μM | 946.2 nM to 1.4 μM | 72.1 nM | 51.1 nM to 101.3 nM |
| 3-7 | SAEPFGTMRFamide | 438.6 nM | 351.3 nM to 530.1 nM | 39.5 nM | 28.5 nM to 54.0 nM |
| 3-8 | SADDSAPFGTMRFamide | N/A | N/A | 1.6 μM | 1.1 μM to 2.4 μM |
| 3-9 | NPENDTPFGTMRFamide | 760.1 nM | 624.1 nM to 923.3 nM | 88.3 nM | 65.2 nM to 119.6 nM |

What is claimed is:

1. A method for high throughput screening for elucidating function of at least one neuropeptide, the method comprising:
identifying the at least one neuropeptide and recombining a nucleic acid sequence of the neuropeptide to obtain a recombinant nucleic acid neuropeptide;
cloning the recombinant nucleic acid neuropeptide into a plasmid to obtain a recombinant neuropeptide plasmid and transforming the recombinant neuropeptide plasmid into a bacterial cell to obtain a transformed neuropeptide bacterial feed; and
feeding the bacterial feed to a subject and observing response of the subject to at least one stimulus thereby elucidating function of the neuropeptide.

2. The method according to claim 1, wherein recombining further comprises adding cleavage sites at 5′ end and 3′ end of the nucleic acid sequence of the neuropeptide.

3. The method according to claim 1, wherein the plasmid further comprises a promoter sequence before the nucleic acid sequence of the neuropeptide.

4. The method according to claim 1 further comprising rescuing the subject from loss of function by feeding the neuropeptide bacterial feed.

5. The method according to claim 1, wherein feeding further comprises delivering mRNA to the subject.

6. The method according to claim 5 further comprising after feeding, translating the mRNA to neuropeptide in the subject.

7. The method according to claim 1, the bacterial cell is at least one Escherichia coli strain selected from: DH5α, and OP50.

8. The method according to claim 1, the plasmid is at least one selected from pDEST-527, and pL4440.

9. The method according to claim 1 further comprising recombining a control sequence, cloning the control sequence in another plasmid, transforming the plasmid into bacterial cell, and feeding the bacterial cell to the subject as a negative control.

10. The method according to claim 1, the neuropeptide is at least one selected from: TRH-1A, TRH-1B, INS-6, PDF-1A, PDF-1B, flp-3, npr-10, frpr-16, GFP, and FLP.

11. A method for rescuing subject by loss of function of at least one neuropeptide, the method comprising:
identifying the at least one neuropeptide and recombining a nucleic acid sequence of the neuropeptide to obtain a recombinant nucleic acid neuropeptide;
cloning the recombinant nucleic acid neuropeptide into a plasmid to obtain a recombinant neuropeptide plasmid and transforming the recombinant neuropeptide plasmid into a bacterial cell to obtain a transformed neuropeptide bacterial feed; and
feeding the bacterial feed to the subject thereby rescuing the subject from loss of function of the neuropeptide.

12. A kit to validate a neuropeptide function using a rescue by feeding assay, the kit comprising: a bacterial feed comprising bacterial cells transformed with a recombinant nucleic acid sequence of the neuropeptide cloned in a plasmid;
instructions for use; and a container.
